(19) United States
(12) Patent Application Publication
(10) Pub. No.: US 2012/0046357 A1
(43) Pub. Date: Feb. 23, 2012

(54) Benzamide and Napthamide Derivatives Inhibiting Nuclear Factor-Kappa (B) - (NK-kB)

(75) Inventors: Jie Zhang, Baltimore, MD (US); Drago Robert Sliskovic, Chelsea, MI (US); Charles E. Ducker, York, PA (US)

(73) Assignee: Profectus Biosciences, Inc., Baltimore, MD (US)

(21) Appl. No.: 13/318,151

(22) PCT Filed: Apr. 29, 2010
(86) PCT No.: PCT/US10/32880

§ 371 Date: Oct. 31, 2011

Related U.S. Application Data
(60) Provisional application No. 61/174,931, filed on May 1, 2009.

(51) Int. Cl. A61K 31/336; A61P 35/00; A61P 9/00; A61P 37/00; A61P 3/00; A61P 31/00; C07D 303/12; A61P 29/00

(52) U.S. Cl. 514/475; 549/546

(57) ABSTRACT

The invention relates to compounds of formula (1) and formula (2),

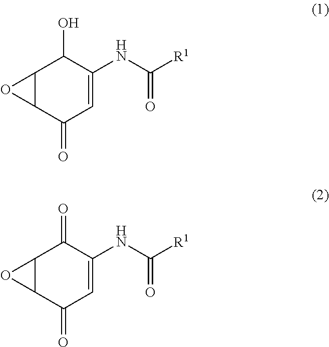

and pharmaceutically acceptable salts thereof for the treatment of cancer, inflammation, auto-immune diseases, diabetes and diabetic complications, infection, cardiovascular disease and ischemia-reperfusion injuries, wherein R¹ is defined herein.

### FIELD OF INVENTION

The invention relates to compounds of formula (1) and (2),

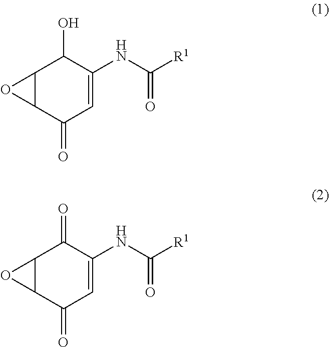

and pharmaceutically acceptable salts thereof, where R¹ is described herein, for the treatment of cancer, inflammation, auto-immune diseases, diabetes and diabetic complications, infection, cardiovascular disease and ischemia-reperfusion injuries.

### BACKGROUND OF INVENTION

Nuclear factor-kappaB (NF-κB) activation has been implicated in a wide variety of diseases, including cancer, diabetes mellitus, cardiovascular diseases, autoimmune diseases, viral replication, septic shock, neurodegenerative disorders, ataxia telangiectasia (AT), arthritis, asthma, inflammatory bowel disease, and other inflammatory conditions. For example, activation of NF-κB by the Gram-negative bacterial lipopolysaccharide (LPS) may contribute to the development of septic shock because NF-κB over-activates transcription of numerous cytokines and modifying enzymes, whose prolonged expression can negatively affect the function of vital organs such as the heart and liver (Arcaroli et al., 2006; Niu et al., 2008).

Similarly, autoimmune diseases such as systemic lupus erythematosus may also involve activation of NF-κB. The NF-κB transcription factor is critical for proper dendritic cell maturation, the loss of which is the hallmark of systemic lupus erythematosus (Kalergis et al., 2008; Kurylowicz & Nauman, 2008). Additionally, in chronic Alzheimer's disease, the amyloid β peptide causes production of reactive oxygen intermediates and indirectly activates gene expression through NF-κB sites (Giri et al., 2005).

Destructive erosion of bone or osteolysis is a major complication of inflammatory conditions such as rheumatoid arthritis (RA), periodontal disease, and periprosthetic osteolysis. RA is an autoimmune disease that affects approximately 1.0% of US adults, with a female to male ratio of 2.5 to 1 (Lawrence et al., 1998). Its hallmark is progressive joint destruction which causes major morbidity. Periodontal disease is highly prevalent and can affect up to 90% of the world's population. It is well known as the leading cause of tooth loss in adults (Pihlstrom et al., 2005). Despite its prevalence, little is known about the mechanism by which periodontal bone erosion occurs, although host response to pathogenic microorganisms present in the mouth appears to trigger the process. Periprosthetic osteolysis is caused by chronic bone resorption around exogenous implant devices until fixation is lost (Harris, 1995), and is considered as resulting from an innate immune response to wear-debris particles, with little contribution by components of the acquired immune system (Goldring et al., 1986).

Although these conditions are initiated by distinct causes and progress by alternative pathways, the important common factor(s) in the pathological process of these diseases are over-production of proinflammatory cytokines which is driven by the constitutive activation of the NF-κB pathway in the inflamed tissue. The bone erosion seen in these conditions is largely localized to the inflamed tissues, distinct from systemic, hormonally regulated bone pathologies, such as osteoporosis. These inflamed tissues, found in many of these diseases, also produce proinflammatory cytokines, i.e., TNF-α, IL-1, and IL-6, that are, in turn, involved in osteoclast differentiation signaling and bone-resorbing activities. Thus, inflammatory osteolysis is the product of enhanced osteoclast recruitment and activation prompted by NF-κB driven proinflammatory cytokines in the inflamed tissue.

Inflammatory bowel disease (IBD) encompasses a number of chronic relapsing inflammatory disorders involving the gastrointestinal tract. The two most prevalent forms of IBD, Crohn's disease and ulcerative colitis, can be distinguished by unique histopathologies and immune responses (Atreya et al., 2008; Bouma & Strober, 2003). The limited efficacy and potential adverse effects of current treatments leave patients and doctors eager for new treatments to manage the chronic relapsing inflammatory nature of these diseases.

Although the exact aetiologies leading to Crohn's disease and ulcerative colitis remain unknown, they are generally thought to result from an inappropriate and ongoing activation of the mucosal immunesystem against the normal luminal flora (Tilg et al., 2008). As a result, resident macrophages, dendritic cells and T cells are activated and begin to secrete predominantly NF-κB-dependent chemokines and cytokines. NF-κB mediated overproduction of key pro-inflammatory mediators is attributed to the initiation and progression of both human IBD and animal models of colitis (Neurath et al., 1998; Wirtz & Neurath, 2007). In particular, macrophages of patients with IBD exhibit high levels of NF-κB DNA binding activity accompanied by increased production of interleukin (IL) 1, IL6 and tumor necrosis factor TNF-α (Neurath et al., 1998). In addition, NF-κB plays a vital role in activating T helper cell 1 (Th1) and T helper cell 2 (Th2) cytokines, both of which are required for promoting and maintaining inflammation (Barnes, 1997). Because of the central role played by NF-κB in IBD, extensive efforts have been made to develop treatments targeting this pathway.

NF-κB has been shown to be constitutively expressed in numerous cancer derived cell lines from breast, ovarian, colon, pancreatic, thyroid, prostate, lung, head and neck, bladder, and skin tumors (Calzado et al., 2007). This has also been seen for B-cell lymphoma, Hodgkin's disease, T-cell lymphoma, adult T-cell leukemia, acute lymphoblastic leukemia, multiple myeloma, chronic lymphocytic leukemia, and acute myelogenous leukemia. NF-κB is a key mediator of normal inflammation as part of the defense response; however, chronic inflammation can lead to cancer, diabetes, and a host of other diseases as mentioned above. Several pro-inflammatory gene products have been identified that mediate a critical role in the carcinogenic process, angiogenesis, invasion, and metastasis of tumor cells. Among these gene products are TNF-α and members of its superfamily, IL-1α, IL-1β, IL-6, IL-8, IL-18, chemokines, MMP-9, VEGF, COX-2, and 5-LOX. The expression of all these genes are mainly regulated by the transcription factor NF-kB, which is constitutively active in most tumors and is induced by carcinogens (such as cigarette smoke), tumor promoters, carcinogenic viral proteins (HIV-tat, KHSV, EBV-LMP1, HTLV 1-tax, HPV, HCV, and HBV), chemotherapeutic agents, and gamma-irradiation (Aggarwal et al., 2006). These observations imply that anti-inflammatory agents that suppress NF-kB should have a potential in both the prevention and treatment of cancer.

The influenza virus protein hemagglutinin also activates NF-κB, and this activation may contribute to viral induction of cytokines and to some of the symptoms associated with influenza (Flory et al., 2000; Pahl & Baeuerle, 1995).

Oxidized lipids from the low density lipoproteins associated with atherosclerosis activate NF-κB, which then activates other genes such as inflammatory cytokines (Liao et al., 1994). Furthermore, mice that are susceptible to atherosclerosis exhibit NF-κB activation when fed an atherogenie diet due to their susceptibility to aortic atherosclerotic lesion formation associated with the accumulation of lipid peroxidation products, induction of inflammatory genes, and the activation of NF-κB transcription factors (Liao et al., 1994). Another important contributor to atherosclerosis is thrombin, which stimulates the proliferation of vascular smooth muscle cells through the activation of NF-κB (Maruyama et al., 1997). A truncated form of IκB repressor protein (IκBα) was shown to be the cause of the hypersensitivity to ionizing radiation and is defective in the regulation of DNA synthesis in ataxia telangiectasia (AT) cells, which have constitutive levels of an NF-κB-activation (Jung et al., 1995). This mutation in the IκBα from the AT cells was shown to inactivate the repressor protein causing the constitutive activation of the NF-kB pathway. In light of all these findings, the abnormal activation or expression of NF-κB is clearly associated with a wide variety of pathologic conditions.

The infection and life-cycle of HIV-1 is tightly coupled to the NF-κB pathway in human mononuclear cells. Viral infection leads to the activation of NF-κB which generates the over stimulation and eventual depletion of T-cells that is the hallmark of AIDS (reviewed in (Argyropoulos & Mouzaki, 2006)). For instance, the expression of CCR5, a key receptor for HIV-1, is regulated by NF-κB (Liu et al., 1998). Deletion analysis of the CCR-5 promoter has demonstrated that loss of the 3′-distal NF-κB/AP-1 site drops transcription by >95% (Liu et al., 1998). These studies would suggest that constitutive repression of NF-κB would cause a dramatic decrease in CCR-5 receptor message. Since HIV-1 entry kinetics are influenced by expressed levels of CCR5 on the target T-cell surface (Ketas et al., 2007; Platt et al., 1998; Reeves et al., 2002), down modulating CCR5 may constrain the expansion of the pool of infected cells that spawns the viral reservoir. CXCR4 expression has also been reported to be influenced by NF-κB (Helbig et al., 2003) suggesting that NF-κB inhibitors may be equally effective against X4-tropic isolates that appear during late-stage infection. NF-κB is required for transcription of the integrated DNA-pro-virus (Baba, 2006; Iordanskiy et al., 2002; Mukeijee et al., 2006; Palmieri et al., 2004; Rizzi et al., 2004; Sui et al., 2006; Williams et al., 2007). In fact, lack of NF-κB activation leads to the generation of a population of cells harboring latent virus which is a major block to eliminating the virus from infected patients (Williams et al., 2006).

NF-κB promotes the expression of over 150 target genes in response to inflammatory stimulators. These genes include; interleukin-1, -2, -6 and the tumor necrosis factor receptor (TNF-R) (these receptors mediate apoptosis, and function as regulators of inflammation), as well as genes encoding immunoreceptors, cell adhesion molecules, and enzymes such as cyclooxygenase-II and inducible nitric oxide synthase (iNOS) (Karin, 2006; Tergaonkar, 2006). It also plays a key role in the progression of diseases associated with viral infections such as HCV and HIV-1.

Members of the NF-κB family include RelA/p65, RelB, c-Rel, p50/p105 (NF-κB1), and p52/p100 (NF-κB2) (Hayden & Ghosh, 2004; Hayden et al., 2006a; Hayden et al., 2006b). The Rel family members function as either homodimers or heterodimers with distinct specificity for cis-binding elements located within the promoter domains of NF-κB-regulated genes (Bosisio et al., 2006; Natoli et al., 2005; Saccani et al., 2004). Classical NF-κB, composed of the RelA/p65 and p50 heterodimer, is the best-studied form of NF-κB (Burstein & Duckett, 2003; Hayden & Ghosh, 2004) and references therein). Prior to cellular stimulation, classical NF-κB resides in the cytoplasm as an inactive complex bound to the IκBα inhibitor proteins. Inducers of NF-κB such as bacterial lipopolysaccharides, inflammatory cytokines, or HIV-1 Vpr protein release active NF-κB from the cytoplasmic complex by activating the hcB-kinase complex (IKK), which phosphorylates hcBa (Greten & Karin, 2004; Hacker & Karin, 2006; Israel, 2000; Karin, 1999; Scheidereit, 2006). Phosphorylation of IκB marks it for subsequent ubiquitinylation and degradation by the 26S proteosome. Free NF-κB dimers translocate into the nucleus where they stimulate the transcription of their target genes.

The molecular design of racemic dehydroxymethylepoxyquinomicin (DHMEQ) was based on the antibiotic epoxyquinomicin C isolated from Amycolatopsis (Chaicharoenpong et al. 2002). DHMEQ was synthesized as a racemate from 2,5-dimethoxyaniline in five steps. Separation of the enantiomers on a chiral column produced both (+) and (−) enantiomers. The (−)-enantiomer was shown to be more potent at inhibiting NF-κB than the (+)-enantiomer (Umezawa et al. 2004). DHMEQ has been characterized to specifically inhibit the translocation of NF-κB into the nucleus (Ariga et cd. 2002). Specifically, it covalently modifies specific cysteine residues in p65 and other Rel homology proteins with a 1:1 stoichiometry ration (Yammamoto et al. 2008). As an NF-κB inhibitor, DHMEQ has been tested extensively in various animal models of diseases and demonstrated a broad spectrum of efficacy including treating solid tumors, hematological malignancies, arthritis, bowel ischemia, and atherosclerosis (Watanabe et al. 2006). Thus, DHMEQ may be useful as a novel treatment for cancer and inflammation (Takeuchi et al. 2003).

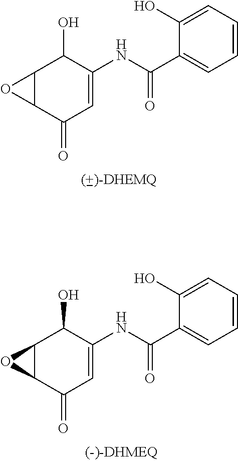

### SUMMARY OF THE INVENTION

The present invention relates to compounds having the structure of formula (1) or formula (2)

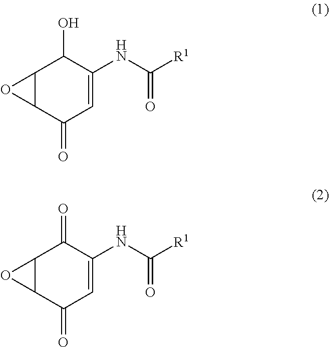

or a pharmaceutically acceptable salt thereof,
wherein

### R¹ is

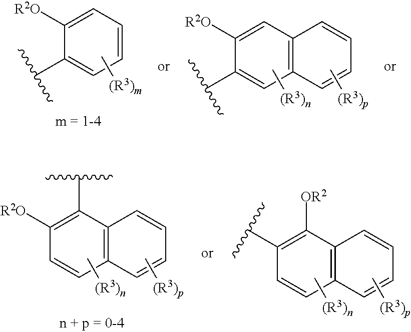

Each R³ is independently hydrogen; CF₃; phenyl optionally substituted with cyano, halo, nitro, hydroxyl, (C1-C6)alkyl, (C1-C6)alkyl-OH, (C1-C6)alkoxy, COR⁴, NR⁵R⁶ or NHCO(C1-C6) alkyl); cyano; halo; nitro; hydroxyl; (C1-C6)alkyl; (C3-C6)cycloalkyl; (C1-C6)alkyl-OH; (C1-C6)-alkyl-NR⁵R⁶; trifluoromethyl; (C1-C6)alkoxy; (C1-C6)thioalkoxy; phenoxy; COR⁴; NR⁵R⁶; NHCO(C1-C6) alkyl; SO₃H; SO₂(C1-C6) alkyl or SO₂NR⁵R⁶, wherein at least one R³ must be other than hydrogen when R¹ is phenyl.

R² is H, R⁷, COR⁷, CONHR⁷, COOR⁷, CH₂OCOR⁷, P(O)(OH)₂, P(O)(O(C1-C6)alkyl)₂, P(O)(OCH₂OCO(C1-C6)alkyl)₂, P(O)(OH)(OCH₂OCO(C1-C6)alkyl), P(O)(OH)(OC1-C6)alkyl), or P(O)(OH)(C1-C6)alkyl), an inorganic salt of P(O)(O(C1-C6)alkyl)₂, P(O)(OCH₂OCO(C1-C6)alkyl)₂, P(O)(OH)(OCH₂OCO(C1-C6)alkyl), P(O)(OH)(OC1-C6)alkyl), or P(O)(OH)(C1-C6)alkyl), or glycosyl (the radical resulting from the removal of a hydroxyl group of the hemiacetal form of a carbohydrate), wherein R⁷ is C1-C6 alkyl, trifluoromethyl, (C3-C6)cycloalkyl, cyclohexylmethyl or phenyl, wherein the phenyl is substituted with 0 to 4 groups selected from fluorine, chlorine, bromine, hydroxyl, trifluoromethyl, (C1-C4)alkyl, (C1-C4)alkoxy, phenylmethyl, phenylmethyl substituted with fluorine, chlorine, bromine, hydroxyl, trifluoromethyl, (C1-C4)alkyl, (C1-C4)alkoxy, 2-, 3-, or 4-pyridinyl, 2-, 4-, or 5-pyrimidinyl.

R⁴ is independently hydroxyl, (C1-C6)alkoxy, phenoxy or —NR⁵R⁶.

Each R⁵ and R⁶ is independently hydrogen; (C1-C6)alkyl or (C3-C6)cycloalkyl; or R⁵ and R⁶ may optionally combine, along with the nitrogen atom, to form a six-membered ring containing an additional heteroatom selected from nitrogen, oxygen or sulfur, wherein the ring is optionally substituted with (C1-C6)alkyl or (C3-C6)cycloalkyl).

m=1 to 4; and n+p=0 to 6.

The present invention also relates to a pharmaceutical composition comprising a compound of formula (1) or formula (2), or a pharmaceutically acceptable salt thereof, and a pharmaceutically acceptable carrier.

The present invention also relates to compounds having the structure of formula (3)

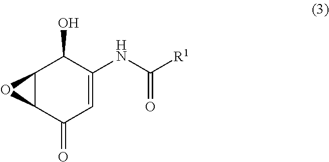

wherein R¹ is as defined above for formula (1) and formula (2).

The present invention further relates to a method of treating cancer, inflammation, auto-immune disease, diabetes and diabetic complications, infection, cardiovascular disease and ischemia-reperfusion injuries, comprising administering to a mammal in need of such treatment, such as a human, a therapeutically effective amount of a compound of formula (1), formula (2) or formula (3), or a pharmaceutically acceptable salt thereof.

The present invention additionally relates to a method of inhibiting gene expression and signal transduction directly or indirectly through the NF-kB pathway in a mammal, such as a human, comprising administering to a mammal in need of such a treatment a therapeutically effective amount of a compound of formula (1), formula (2) or formula (3), or a pharmaceutically acceptable salt thereof.

### BRIEF DESCRIPTION OF THE FIGURES

The figures represent specific embodiments of the described invention and are therefore used for illustrative purposes only. Accordingly, the figures are not intended to limit the scope of the invention.

FIG. 1 shows the concentration dependent inhibition of NF-κB driven expression of luciferase (A) and GFP (B) by the compound of Example 10.

FIG. 2 shows that the compound of Example 10 inhibits p65 (RelA) binding to DNA in the canonical pathway.

FIG. 3 shows that the compound of Example 10 inhibits RelB binding to DNA in the non-canonical pathway.

FIG. 4 shows that the compound of Example 10 inhibits c-Rel binding to DNA in the NF-kB pathway.

FIG. 5 shows IL-6 expression from RAW264.7 cells, and in particular, the effect of the compound of Example 10 on IL-6 secretion from RAW264.7 cells stimulated by 1 μg/mL of LPS. The cells were treated with the indicated concentrations of the compound of Example 10 for 2 hours, and then LPS was added and incubation continued for 4 hours before mIL-6 level in media was determined by ELISA.

FIG. 6 shows growth inhibition of RPMI8226 multiple myeloma cells by the compound of Example 10.

FIG. 7 shows growth inhibition of U266B1 multiple myeloma cells by a compound of Example 10.

### DETAILED DESCRIPTION

### Definitions

The terms used to describe the present invention have the following meanings herein. The compounds and intermediates of the present invention may be named according to either the IUPAC (International Union for Pure and Applied Chemistry) or CAS (Chemical Abstracts Service) nomenclature systems.

The carbon atom content of the various hydrocarbon-containing moieties herein may be indicated by a prefix designating the minimum and maximum number of carbon atoms in the moiety, for example, the prefix (Ca-Cb)alkyl indicate an alkyl moiety of the integer “a” to “b” carbon atoms, inclusive. Thus, for example, (C1-C6)alkyl refers to an alkyl group of one to six carbon atoms inclusive. The term “alkyl” denotes a straight or branched chain of carbon atoms with only hydrogen atom substituents, wherein the carbon chain optionally contains one or more double or triple bonds, or a combination of double bonds and triple bonds. Examples of alkyl groups include, but are not limited to, methyl, ethyl, propyl, isopropyl, propenyl, propynyl, hexadienyl, and the like.

The term “alkoxy” refers to straight or branched, monovalent, saturated aliphatic chains of carbon atoms wherein one of the carbon atoms has been replaced with an oxygen atom. Examples of alkoxy groups include, but are not limited to, methoxy, ethoxy and iso-propoxy.

The term “cycloalkyl” refers to a saturated and optionally unsaturated monocyclic or bicyclic arrangement of aliphatic chains. Examples of cycloalkyl groups include, but are not limited to, cyclopropyl, cyclobutyl, cyclopentyl and cyclohexene. Cycloalkyl groups may also be optionally fused to aromatic hydrocarbons such as benzene to form fused cycloalkyl groups, such as indanyl and the like.

The term “halo” refers to chloro, bromo, fluoro, or iodo.

The term “substituted” refers to a hydrogen atom on a molecule has been replaced with a different atom or molecule. The atom or molecule replacing the hydrogen atom is denoted as a “substituent.”

The phrase “therapeutically effective amount” refers to an amount of a compound that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition.

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “mammal” relates to an individual animal that is a member of the taxonomic class Mammalia. Examples of mammals include, but are not limited to, humans, dogs, cats, horses and cattle. In the present invention, the preferred mammal is a human.

In an exemplary embodiment, the compounds of the present invention have the structure shown in formula (3), wherein R¹ is as defined above for the compounds of formula (1) and formula (2).

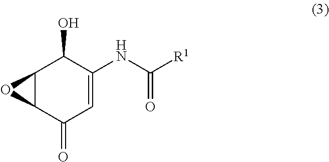

The compounds may be resolved into their pure enantiomers by methods known to those skilled in the art, for example by formation of diastereoisomeric salts which may be separated, for example, by crystallization; formation of diastereoisomeric derivatives or complexes which may be separated (for example, by crystallization, gas-liquid or liquid chromatography); selective reaction of one enantiomer with an enantiomer-specific reagent (for example, enzymatic esterification); or gas-liquid or liquid chromatography in a chiral environment, for example, on a chiral support for example silica with a bound chiral ligand or in the presence of a chiral solvent. It will be appreciated that where the desired stereoisomer is converted into another chemical entity by one of the separation procedures described above, a further step is required to liberate the desired enantiomeric form. Alternatively, the specific stereoisomers may be synthesized by using an optically active starting material, by asymmetric synthesis using optically active reagents, substrates, catalysts or solvents, or by converting one stereoisomer into the other by asymmetric transformation.

Wherein the compounds contain one or more additional stereogenic centers, those skilled in the art will appreciate that all diastereoisomers and diastereoisomeric mixtures of the compounds illustrated and discussed herein are within the scope of the present invention. These diastereoisomers may be islolated by methods known to those skilled in the art, for example, by crystallization, gas-liquid or liquid chromatography. Alternatively, intermediates in the course of the synthesis may exist as racemic mixtures and be subjected to resolution by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example, enzymatic esterification; or gas-liquid or liquid chromatography in a chiral environment, for example, on a chiral support for example silica with a bound chiral ligand or in the presence of a chiral solvent. It will be appreciated that where the desired stereoisomer is converted into another chemical entity by one of the separation procedures described above, a further step is required to liberate the desired enantiomeric form. Alternatively, the specific stereoisomers may be synthesized by using an optically active starting material, by asymmetric synthesis using optically active reagents, substrates, catalysts or solvents, or by converting one stereoisomer into the other by asymmetric transformation. These methods are described in more detail in texts such as “Chiral Drugs”, Cynthia A. Challener (Editor), Wiley, 2002 or “Chiral Drug Separation” by Bingyunh Li and Donald T. Haynia in “Encyclopedia of Chemical Processing” by Sunggyu Lee and Lee Lee (Editors), CRC Press, 2005.

The compounds of the present invention, and the salts thereof, may exist in the unsolvated as well as the solvated forms with pharmaceutically acceptable solvents such as water, ethanol, and the like.

Selected compounds of formula (1) and formula (2) and their salts and solvates may exist in more than one crystal form. Polymorphs of compounds represented by formula (1) and formula (2) form part of this invention and may be prepared by crystallization of a compound of formula (1) and formula (2) under different conditions. For example, using different solvents or solvent mixtures for recrystallization; crystallization at different temperatures; various modes of cooling, ranging from very fast to very slow cooling during crystallization. Polymorphs may also be obtained by heating or melting a compound of formula (1) and formula (2) followed by gradual or fast cooling. The presence of polymorphs may be determined by solid state NMR spectroscopy, IR spectroscopy, differential scanning calorimetry, powder X-ray diffraction or other such techniques.

This invention also includes isotopically-labeled compounds, which are identical to those described by formulas (1) and (2), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, sulfur and fluorine, such as ²H, ³H, ¹³C, ¹⁴C, ¹⁵N, ¹⁸O, ¹⁷O, ³⁵S, ³⁶Cl, ¹²⁵I, ¹²⁹I and ¹⁸F respectively. Compounds of the present invention and pharmaceutically acceptable salts of the compounds which contain the aforementioned isotopes and/or other isotopes of other atoms are within the scope of this invention. Certain isotopically-labeled compounds of the present invention, for example those into which an isotope such as ²H(deuterium) are incorporated can afford certain therapeutic advantage resulting from greater metabolic stability, for example, increased in vivo half life or reduced dosage requirements and, hence, may be preferred in some circumstances. Isotopically labeled compounds of formulae (1) and (2) of this invention, salts and solvates thereof can generally be prepared by carrying out procedures disclosed in the schemes and/or in the Examples below, by substituting a readily available isotopically labeled reagent for a non-isotopically labeled reagent.

Pharmaceutically acceptable salts, as used herein in relation to compounds of the present invention, include pharmaceutically acceptable inorganic and organic salts of said compounds. These salts can be prepared in situ during the final isolation and purification of a compound, or by separately reacting the compound with a suitable organic or inorganic acid and isolating the salt thus formed. Representative salts include, but are not limited to, the hydrobromide, hydrochloride, hydroiodide, sulfate, bisulfate, nitrate, acetate, trifluoroacetate, oxalate, besylate, camsylate, palmitate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. Compounds of the present invention may also react to form salts with pharmaceutically acceptable metal and amine cations formed from organic and inorganic bases. The term “pharmaceutically acceptable metal cation” contemplates positively charged metal ions derived from sodium, potassium, calcium, magnesium, aluminum, iron, zinc and the like. The term “pharmaceutically acceptable amine cation” contemplates the positively charged ions derived from ammonia and organic nitrogenous bases strong enough to form such cations. Bases useful for the formation of pharmaceutically acceptable nontoxic base addition salts of compounds of the present invention form a class whose limits are readily understood by those skilled in the art. (See, for example, Berge, et al., “Pharmaceutical Salts,” J. Pharm. Sci., 66:1-19 (1977)).

The present invention further includes prodrugs of compounds of formula (1) and formula (2). A prodrug of a compound of formula (1) and formula (2) may be formed in a conventional manner with a functional group of the compound, such as with an amino, hydroxy or carboxy group. The term “prodrug” means a compound that is transformed in vivo to yield a compound of formula (1) and formula (2) or a pharmaceutically acceptable salt or solvate of the compound. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987. For example, if a compound of the present invention contains an alcohol functional group, a prodrug can be formed by the replacement of the hydrogen atom of the alcohol group with a group such as COR⁷ providing an ester prodrug, CONHR⁷ providing a carbamate prodrug, COOR⁷ providing a carbonate prodrug, CH₂OCOR⁷ providing and alkylcarbonyloxymethyl prodrug, P(O)(OH), providing a phosphate prodrug, P(O)(O(C1-C6)alkyl)₂ providing a phosphate prodrug, P(O)(OCH₂OCO(C1-C6)alkyl)₂ providing a phosphate prodrug, P(O)(OH)(OCH₂OCO(C1-C6)alkyl) providing a phosphate prodrug, P(O)(OH)(OC1-C6)alkyl) providing a phosphate prodrug, or P(O)(OH)(C1-C6)alkyl) providing a phosphonate prodrug, and the corresponding inorganic salts of the phosphate and phosphonate prodrugs, or glycosyl (the radical resulting from the removal of a hydroxyl group of the hemiacetal form of a carbohydrate), wherein R⁷ is C1-C6 alkyl, trifluoromethyl, cyclopropyl, cyclohexyl, cyclohexylmethyl, phenyl, phenyl substituted with fluorine, chlorine, bromine, hydroxyl, trifluoromethyl, (C1-C4)alkyl, (C1-C4)alkoxy, phenylmethyl, phenylmethyl substituted with fluorine, chlorine, bromine, hydroxyl, trifluoromethyl, (C1-C4)alkyl, (C1-C4) alkoxy, 2-, 3-, or 4-pyridinyl, or 2-, 4-, or 5-pyrimidinyl.

In an exemplary embodiment, R¹ is

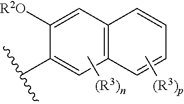

where R², R³, n and p are as defined above.

In an exemplary embodiment, R¹ is

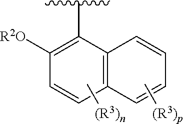

where R², R³, n and p are as defined above.

In an exemplary embodiment, R¹ is

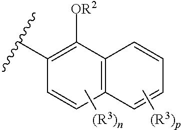

where R², R³, n and p are as defined above.

In an exemplary embodiment, R¹ is

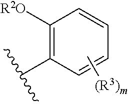

where R², R³ and m are as defined above.

In general, compounds of the present invention where R¹ represents the moieties shown below

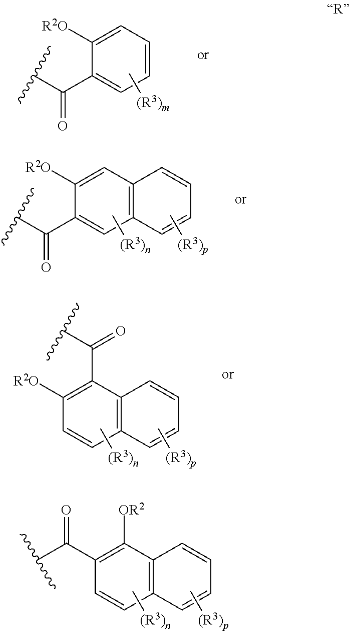

as described herein, are prepared by the general synthetic methods outlined in Reaction Schemes 1 and 2.

In an exemplary embodiment, NR⁵R⁶ is morpholine, thiomorpholine or piperazine.

Referring to Reaction Scheme 1, compounds 2-5 can be prepared according to literature procedures (Taylor et al., Synthesis 1998, 775). Treatment of 2,5-dimethoxyaniline 1 with di-tert-butyl dicarbonate (Boc₂O) and triethylamine in methanol or tetrahydrofuran at temperatures ranging from 0° C. to room temperature gave the protected aniline derivative 2. Oxidation with bis(acetoxyiodo) benzene in methanol at 0° C. gave the ketal 3. Monoepoxidation to yield 4 was achieved using 30% aqueous hydrogen peroxide and a base such as aqueous sodium hydroxide or potassium carbonate in aqueous tetrahydrofuran at temperatures ranging from 0° C. to room temperature. Selective removal of the Boc group with a 4/1 dichloromethane/trifluoroacetic acid mixture at temperatures ranging from 0° C. to room temperature gave the free amine 5. Alternatively, this deprotection can be achieved using boron trifluoride-diethyl ether complex and activated molecular sieves in a solvent such as dichloromethane at room temperature. The amine 5 was then coupled with an acid chloride (RC1) using a base such as lithium tert-butoxide (LiO^{t}Bu) in a solvent such as anhydrous tetrahydrofuran at −78° C. to give the ketal 6. The various acid chlorides (RC1) were prepared from the corresponding carboxylic acid by refluxing in neat thionyl chloride. The ketal 6 was de-protected in an acidic media such as trifluoroacetic acid in a solvent such as dichloromethane at temperatures ranging from 0° C. to room temperature to give diketone 7. Regioselective reduction of 7 was achieved by treatment with a slight excess of a mild reducing agent such as sodium triacetoxyborohydride (NaBH(OAc)₃) in a solvent such as methanol at temperatures ranging from 0° C. to room temperature.

An alternative synthetic route is depicted in Reaction Scheme 2. Treatment of 2,5-line 1 with an acid chloride (RC1) and abase such as pyridine in a solvent such as anhydrous tetrahydrofuran at temperatures ranging from 0° C. to room temperature gave 10. Oxidation with bis(acetoxyiodo) benzene in methanol at 0° C. gave the ketal 11. Monoepoxidation to yield 6 was achieved using 30% aqueous hydrogen peroxide and a base such as aqueous sodium hydroxide at temperatures ranging from 0° C. to room temperature. The ketal 6 was deprotected in an acidic media such as trifluoroacetic acid in a solvent such as dichloromethane at temperatures ranging from 0° C. to room temperature to give diketone 7. Regioselective reduction of 7 was achieved by treatment with a slight excess of a mild reducing agent such as sodium triacetoxyborohydride (NaBH(OAc)₃) in a solvent such as methanol at temperatures ranging from 0° C. to room temperature.

A variation on Scheme 1 is shown in Scheme 3. In this case the hydroxyl group is O-acetylated by treatment of the appropriate salicylic acid 12 with acetic anhydride and an acid such as sulfuric acid. This acetylated product is then treated with oxalyl chloride in a solvent such as dichloromethane to give the corresponding acid chloride 13. This acid chloride is then reacted with the free amine 5 using a base such as lithium tert-butoxide (LiO^{t}Bu) in a solvent such as anhydrous tetrahydrofuran at −78° C. to give the O-acetylated ketal. Treatment of the O-acetylated ketal with a base such as potassium carbonate in a solvent such as aqueous methanol gave the deprotected ketal 6.

It was discovered that the diketone intermediates of the general formula (7) as depicted in Reaction Schemes (1) and (2) also exhibited significant activity as NF-κB inhibitors. Thus, diketones of general formula (2) as described herein are considered as part of the invention.

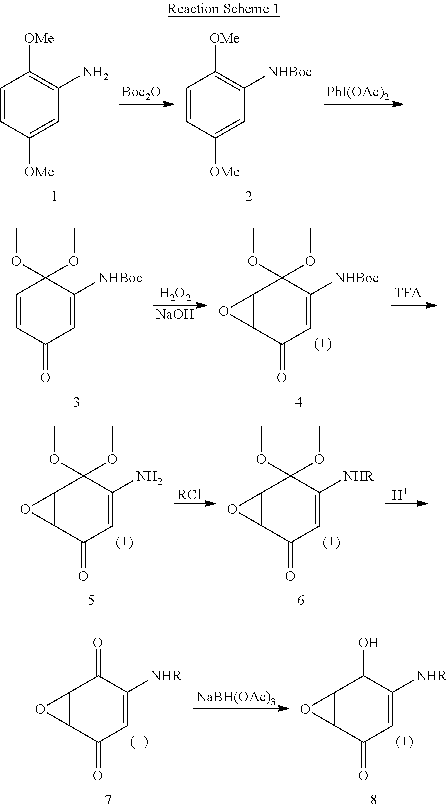

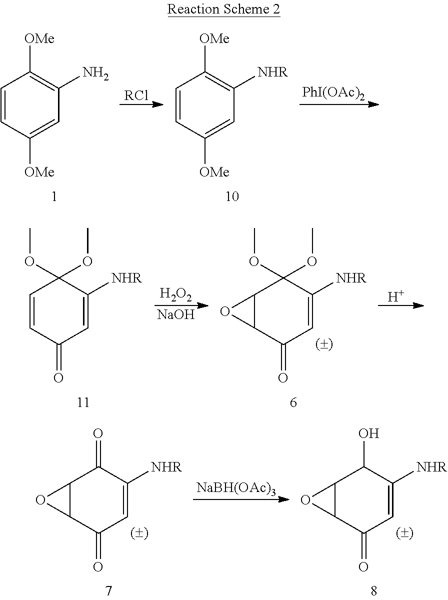

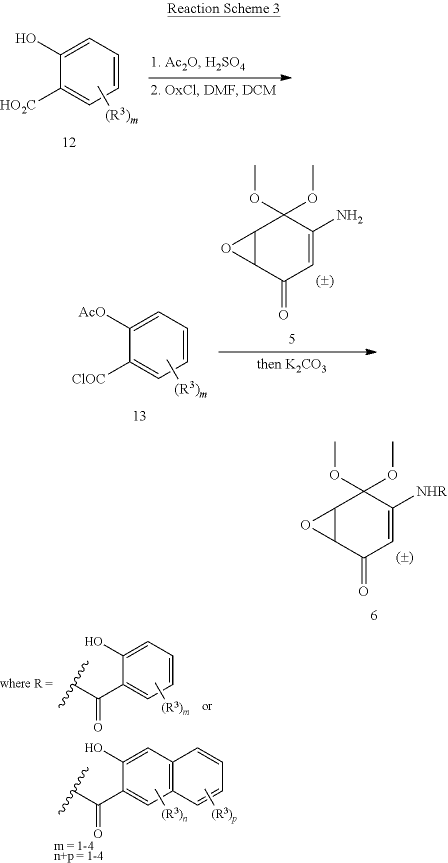

A pharmaceutical composition of the present invention comprises a therapeutically effective amount of a compound of formula (1), or a pharmaceutically acceptable salt thereof, and a pharmaceutically acceptable carrier, vehicle, diluents or excipient. A preferred pharmaceutical composition of the present invention comprises a therapeutically effective amount of a compound of formula (2), or a pharmaceutically acceptable salt thereof, and a pharmaceutically acceptable carrier, vehicle, diluents or excipient. The pharmaceutical compositions formed by combining the compounds of this invention and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like.

Thus, for purposes of oral administration, tablets containing various excipients such as sodium citrate, calcium carbonate and/or calcium phosphate, may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions of elixirs are desired for oral administration, the active pharmaceutical agent therein may be combined with various sweetening of flavoring agents, coloring matter or dyes and, if desired, emulsifying or suspending agents, together with diluents such as water, ethanol, propylene glycol, glycerin and/or combinations thereof.

For parenteral administration, solutions of the compounds or compositions of this invention in sesame or peanut oil, aqueous propylene glycol, or in sterile aqueous solutions may be employed. Such aqueous solutions should be suitably buffered if necessary and the liquid diluents first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

In an exemplary embodiment, the pharmaceutical preparation is in unit dosage form. In such form, the preparation is subdivided into unit doses containing appropriate quantities of the active component. The unit dosage form can be a packaged preparation, for example, packeted tablets, capsules, and powders in vial or ampoules. The unit dosage form can also be a capsule, cahet, or tablet itself or it can be the appropriate number of any of these packaged forms.

Methods of preparing various pharmaceutical compositions with a certain amount of active ingredient are known to those skilled in the art. For examples of methods of preparing pharmaceutical compositions, see Remington: The Science and Practice of Pharmacy, Lippincott, Williams & Wilkins, 21^{st} ed. (2005), which is incorporated by reference in its entirety.

### EXAMPLES

### Example 1

### 2-hydroxy-N-(2-hydroxy-5-oxo-7-oxabicyclo[4.1.0]hept-3-en-3-yl)-4-(trifluoromethyl)benzamide (8a)

### A. Preparation of tert-butyl 2,5-dimethoxyphenylcarbamate (2)

To a solution of 2,5-dimethoxyaniline 1 (50 g, 326 mmol) in MeOH (1 L) in an ice bath under an inert nitrogen atmosphere was added triethylamine (55 mL, 397 mmol), followed by the drop-wise addition of Boc₂O (78 g, 359 mmol) in methanol (150 mL). The reaction was stirred overnight. After judging incomplete by thin layer chromatography, additional Boc₂O (22 g, 69 mmol) and triethylamine (55 mL, 397 mmol) was added and the solution was stirred 3 days. The methanol was removed and the residue was dissolved in ethyl acetate, which was rinsed with diluted hydrochloric acid (2×) and brine before drying over anhydrous magnesium sulfate, followed by filtration, and solvent evaporation to afford 50 g (61%) tert-butyl 2,5-dimethoxyphenylcarbamate (2) as a brown oil. The ¹H NMR was consistent with that reported in the literature (Synthesis 1998, 775).

### B. Preparation of tert-butyl 6,6-dimethoxy-3-oxocyclohexa-1,4-dienylcarbamate (3)

A methanolic (700 mL) solution of compound 2 (29 g, 115 mmol) was cooled in an ice bath before the addition of bis(acetoxyiodo) benzene (62 g, 194 mmol) in six portions over a period of 30 minutes. The solution was stirred in the ice bath for 2 hours then brought to room temperature and stirred overnight. The reaction mixture was diluted with ethyl acetate (1.5 L) and rinsed with water, dilute hydrochloric acid, and brine. The aqueous was back-extracted once with ethyl acetate and the organics combined before drying over anhydrous magnesium sulfate, followed by filtration, and solvent evaporation. The resulting liquid was purified over silica gel using a gradient of ethyl acetate (0 to 2%) in heptane, giving 6.9 g (21%) (3) as a yellow-orange solid. Material was sufficiently pure (approx. 95%) by ¹H NMR, which was consistent with that reported in the literature (Synthesis 1998, 775).

### C. Preparation of tert-butyl 2,2-dimethoxy-5-oxo-7-oxabicyclo[4.1.0]hept-3-en-3-ylcarbamate (4)

Compound 3 (3.4 g, 12.7 mol) in tetrahydrofuran (93 mL) was cooled in an ice bath. To the stirring solution, in drop-wise fashion, was added hydrogen peroxide (30% aq., 22 mL) and aqueous sodium hydroxide (1M, 61 mL) in tandem using 2 separate addition funnels and utilizing a Claisen adapter. The reaction mixture was stirred for 30 minutes in the ice bath and then for 5 hours at room temperature. The flask was cooled in an ice bath and the peroxide was carefully quenched with manganese dioxide. After filtration of the mixture over a small bed of silica gel followed by rinsing of the silica gel with ethyl acetate, the mixture was washed with brine. The organics were then dried over anhydrous magnesium sulfate, filtered, and evaporated. The resulting oil was treated with pentane to precipitate an off white solid after solvent evaporation yielding 2 g (56%) of compound (4). ¹H NMR indicated 20% starting material in the mixture, which was easily removed in the next step (treatment with TFA). The ¹H NMR of the product was consistent with that reported in the literature (Synthesis 1998, 775).

### D. Preparation of 4-amino-5,5-dimethoxy-7-oxabicyclo[4.1.0]hept-3-en-2-one (5)

A solution of compound 4 (300 mg, 1.1 mmol) in anhydrous dichloromethane (6 was stirred in an ice bath under an inert nitrogen atmosphere. To this solution was added trifluoroacetic acid (1.5 mL) drop-wise and the solution was brought to room temperature, stirring 3 hours. After judging complete by thin layer chromatography, the solvents were evaporated and the residue was dissolved in ethyl acetate. Solid sodium bicarbonate (2 g) was carefully added and the solution was stirred 10 minutes. The salt was filtered and rinsed with ethyl acetate before evaporation of the solvent. The crude compound was purified over silica gel using a gradient of ethyl acetate (0 to 100%) in heptane. The product eluted in 100% ethyl acetate giving 178 mg (91%) of compound (5). The ¹H NMR of the product was consistent with that reported in the literature (Synthesis 1998, 775).

### E. Preparation of N-(2,2-dimethoxy-5-oxo-7-oxabicyclo[4.1.0]hept-3-en-3-yl)-2-hydroxy-4-(trifluoromethyl)benzamide (6a)

In a 25-mL round bottom flask, 2-hydroxy-4-(trifluoromethyl)benzoic acid (309 mg, 1.5 mmol) was dissolved in dichloromethane (6.0 mL). The solution was cooled to 0° C. and flushed with nitrogen. To the solution was added oxalyl chloride (209 mg, 1.65 mmol) followed by dimethylformamide (2 drops). The reaction was stirred at 0° C. for 20 min and then allowed to warm to room temperature. Once the solution turned cleared, the volatiles were removed by rotory evaporation. In a 25 mL round bottom flask, epoxyamine (5) (100 mg, 0.54 mmol) was dissolved in tetrahydrofuran (4 mL). The solution was cooled to −78° C. and stirred under nitrogen. After 10 minutes, lithium tert-butoxide (0.81 mL, 0.81 mmol, 1.0 M in tetrahydrofuran) was added and the mixture was stirred at −78° C. for 20 minutes. The acid chloride obtained above (1.5 mmol) was dissolved in 2 mL of tetrahydrofuran and transferred to the reaction mixture via a cannula. The round bottom flask containing the acid chloride was rinsed with tetrahydrofuran (2×0.5 mL). The reaction mixture was stirred at −78° C. for 30 minutes and then stirred at room temperature. Once the reaction was complete by TLC, the mixture was diluted with ethyl acetate and saturated aqueous ammonium chloride. The layers were separated and the organic layer was washed with brine, dried with sodium sulfate, filtered and concentrated to yield a brown oil which was a mixture of the title compound (6a) and O-aroylated compound. The crude mixture of 6a was dissolved in methanol:water (8:1, 5.4 mL) and potassium carbonate (28 mg, 0.2 mmol) was added. The reaction was monitored by LC/NIS and more potassium carbonate (70 mg, 0.5 mmol) was added. Once complete by LC/MS, the reaction was diluted with ethyl acetate and washed with saturated aqueous sodium bicarbonate. The organic layer was separated, washed with brine, dried with sodium sulfate, filtered and concentrated. The crude oil was purified via silica gel chromatography to yield 6a as yellow oil (85 mg, 42%). The product structure was confirmed by ¹H NMR. NMR (CDCl₃): δ11.50 (br.s, 1H), 8.65 (br.s, 1H), 7.50 (m, 1H), 7.35 (m, 1H), 7.25 (m, 2H), 3.85 (m, 1H), 3.70 (s, 3H), 3.60 (m, 1H), 3.35 (s, 3H) ppm

### F. Preparation of N-(2,5-dioxo-7-oxabicyclo[4.1.0]hept-3-en-3-yl)-2-hydroxy-4-(trifluoromethyl)benzamide (7a)

The ketal 6a (85 mg, 0.23 mmol) was dissolved in dichloromethane (1.0 mL) and cooled to 0° C. To this solution, trifluoroacetic acid (0.61 mL) was added slowly. The ice bath was removed and the reaction was stirred at room temperature overnight. After LC/MS confirmed completion of the reaction, the solution was diluted with ethyl acetate and washed with saturated aqueous sodium bicarbonate. The aqueous layer was washed twice with ethyl acetate. The organic layers were combined, washed with water and brine, dried with sodium sulfate, filtered, and concentrated to yield 7a as a brown oil (70 mg, 90%) which was used without further purification. The product structure was confirmed by H NMR. NMR (CDCl₃): δ11.40 (br.s., 1H), 8.85 (br.s., 1H), 7.65 (m, 2H), 7.30 (m, 1H), 7.25 (m, 1H), 4.00 (m, 1H), 3.90 (m, 1H) ppm.

### G. Preparation of (±)-2-hydroxy-N-(2-hydroxy-5-oxo-7-oxabicyclo[4.1.0]hept-3-en-3-yl)-4-(trifluoromethyl)benzamide (8a)

The diketone 7a (65 mg, 0.199 mmol) was dissolved in methanol (1.0 mL). Sodium triacetoxyborohydride (42 mg, 0.199 mmol) was added to the solution and stirred at room temperature. After 30 minutes, LC/MS showed starting material still present so more Sodium triacetoxyborohydride (25 mg, 0.12 mmol) was added. Once the reaction was complete by LC/MS, it was diluted with ethyl acetate and washed with saturated aqueous ammonium chloride. The organic layer was washed with brine, dried with sodium sulfate, filtered and concentrated. A crude brown solid was obtained and some methanol was added. The solution was filtered and 8a was obtained as a white solid (10 mg, 15%). The product structure was confirmed by ¹H NMR. NMR (DMSO-d₆): δ8.00 (m, 1H), 7.25 (m, 1H), 6.95 (m, 1H), 6.75 (m, 1H), 4.80 (m, 1H), 3.80 (m, 1H), 3.45 (m, 1H) ppm.

Employing the general method of Example 1, the following compounds were prepared:

### Example 2 (±)-4-chloro-2-hydroxy-N-(2-hydroxy-5-oxo-7-oxabicyclo[4.1.0]hept-3-en-3-yl)benzamide. The product structure was confirmed by ¹H NMR. NMR (DMSO-d₆): δ7.80 (m, 1H), 6.80 (m, 2H), 6.60 (m, 1H), 4.80 (m, 1H), 3.80 (m, 1H), 3.45 (m, 1H) ppm.

### Example 3

(±)-2-hydroxy-N-(2-hydroxy-5-oxo-7-oxabicyclo[4.1.0]hept-3-en-3-yl)-4-methylbenzamide. The product structure was confirmed by ¹H NMR. NMR (DMSO-d₆): δ7.85 (m, 1H), 6.80 (m, 2H), 6.70 (m, 1H), 4.80 (m, 1H), 3.80 (m, 1H), 3.45 (m, 1H), 2.40 (s, 3H) ppm.

### Example 4

(±)-2-hydroxy-N-(2-hydroxy-5-oxo-7-oxabicyclo[4.1.0]hept-3-en-3-yl)-3-methylbenzamide. The product structure was confirmed by ¹H NMR. NMR (Acetone-d₆): δ7.80 (m, 1H), 7.40 (m, 1H), 6.90 (m, 2H), 5.00 (m, 1H), 3.80 (m, 1H), 3.45 (m, 1H), 2.25 (s, 3H) ppm.

### Example 5

(±)-2-hydroxy-N-(2-hydroxy-5-oxo-7-oxabicyclo[4.1.0]hept-3-en-3-yl)-4-methoxybenzamide. The product structure was confirmed by ¹H NMR. NMR (Acetone-d₆): δ7.85 (m, 1H), 7.00 (m, 1H), 6.60 (m, 2H), 5.00 (m, 1H), 3.90 (m, 1H), 3.85 (s, 3H), 3.40 (m, 1H) ppm.

### Example 6

(±)-3-chloro-2-hydroxy-N-(2-hydroxy-5-oxo-7-oxabicyclo[4.1.0]hept-3-en-3-yl)benzamide. The product structure was confirmed by ¹H NMR. NMR (Acetone-d₆): δ7.90 (m, 1H), 7.55 (m, 1H), 7.00 (m, 1H), 6.90 (m, 1H), 4.80 (m, 1H), 3.90 (m, 1H), 3.40 (m, 1H) ppm.

### Example 7

(±)-3-hydroxy-N-(2-hydroxy-5-oxo-7-oxabicyclo[4.1.0]hept-3-en-3-yl)-2-naphthamide. The product structure was confirmed by ¹H NMR. NMR (Acetone-d₆): δ8.70 (m, 1H), 7.95 (m, 1H), 7.80 (m, 1H), 7.50 (m, 2H), 7.35 (m, 1H), 6.90 (m, 1H), 4.95 (m, 1H), 3.90 (m, 1H), 3.40 (m, 1H) ppm.

### Example 8

(±)-1-hydroxy-N-(2-hydroxy-5-oxo-7-oxabicyclo[4.1.0]hept-3-en-3-yl)-2-naphthamide. ¹H NMR. NMR (DMSO-d₆): δ8.40 (m, 1H), 7.80 (m, 2H), 7.60 (m, 3H), 6.90 (m, 1H), 6.65 (m, 1H), 4.90 (m, 1H), 3.85 (m, 1H), 3.45 (m, 1H) ppm.

### Example 9

(±)-5-bromo-2-hydroxy-N-(2-hydroxy-5-oxo-7-oxabicyclo[4.1.0]hept-3-en-3-yl)benzamide. ¹H NMR. NMR (DMSO-d₆): δ7.90 (m, 1H), 7.50 (m, 1H), 6.95 (m, 1H), 6.90 (m, 1H), 6.65 (m, 1H), 4.80 (m, 1H), 3.85 (m, 1H), 3.45 (m, 1H) ppm.

### Example 10

(±)-4-(dimethylamino)-2-hydroxy-N-(2-hydroxy-5-oxo-7-oxabicyclo[4.1.0]hept-3-en-3-yl)benzamide. ¹H NMR. NMR (DMSO-d₆): δ7.70 (m, 1H), 6.95 (m, 1H), 6.60 (br.s, 1H), 6.30 (m, 1H), 6.00 (m, 1H), 4.75 (m, 1H), 3.85 (m, 1H), 3.45 (m, 1H), 2.90 (s, 6H) ppm.

### Example 11

(±)-3-hydroxy-N-(2-hydroxy-5-oxo-7-oxabicyclo[4.1.0]hept-3-en-3-yl)-7-methoxy-2-naphthamide. ¹H NMR. NMR (DMSO-d₆): δ 11.6 (br.s, 1H), 8.50 (m, 1H), 7.65 (m, 1H), 7.40 (m, 1H), 7.30 (m, 1H), 7.20 (m, 1H), 6.95 (m, 1H), 6.70 (m, 1H), 4.90 (m, 1H), 3.85 (m, 1H), 3.80 (S, 3H), 3.45 (m, 1H) ppm.

### Example 12

(±)-N-(2,5-dioxo-7-oxabicyclo[4.1.0]hept-3-en-3-yl)-2-hydroxy-1-naphthamide. NMR. NMR (DMSO-d₆): δ11.1 (br.s, 1H), 10.3 (br.s, 1H), 8.05 (m, 1H), 7.90 (m, 2H), 7.50 (m, 2H), 7.40 (m, 1H), 7.25 (m, 1H), 4.20 (m, 1H), 4.00 (m, 1H), 3.50 (m, 1H) ppm.

### Example 13

(±)-5-bromo-2-hydroxy-N-(2-hydroxy-5-oxo-7-oxabicyclo[4.1.0]hept-3-en-3-yl)-3-methylbenzamide. NMR. NMR (DMSO-d₆): δ7.95 (m, 1H), 7.50 (m, 1H), 6.95 (m, 1H), 4.80 (m, 1H), 3.85 (m, 1H), 3.45 (m, 1H), 2.25 (s, 3H) ppm.

### Example 14

(±)-5-bromo-N-(2,5-dioxo-7-oxabicyclo[4.1.0]hept-3-en-3-yl)-2-hydroxy-3-methylbenzamide. ¹H NMR. NMR (DMSO-d₆): δ 7.90 (m, 1H), 7.50 (m, 2H), 4.05 (m, 1H), 3.85 (m, 1H), 2.25 (m, 3H) ppm.

### Example 15

(±)-7-bromo-3-hydroxy-N-(2-hydroxy-5-oxo-7-oxabicyclo[4.1.0]hept-3-en-3-yl)-2-naphthamide. ¹H NMR. NMR (DMSO-d₆): δ8.60 (m, 1H), 8.25 (m, 1H), 7.70 (m, 1H), 7.60 (m, 1H), 7.25 (m, 1H), 6.95 (m, 1H), 6.70 (m, 1H), 4.90 (m, 1H), 3.85 (m, 1H), 3.45 (m, 1H) ppm.

### Example 16

(±)-3,4-difluoro-N-(2-hydroxy-5-oxo-7-oxabicyclo[4.1.0]hept-3-en-3-yl)-2-methoxybenzamide. ¹H NMR. NMR (DMSO-d₆): δ10.50 (br.s, 1H), 7.80 (m, 1H), 7.40 (m, 1H), 6.90 (m, 1H), 4.90 (m, 1H), 4.20 (s, 3H), 3.85 (m, 1H), 3.45 (m, 1H) ppm.

### Example 17

(±)-2-hydroxy-N-(2-hydroxy-5-oxo-7-oxabicyclo[4.1.0]hept-3-en-3-yl)-6-isopropyl-3-methylbenzamide. ¹H NMR. NMR (DMSO-d₆): δ 7.10 (m, 1H), 6.95 (m, 1H), 6.80 (m, 1H), 4.75 (m, 1H), 3.85 (m, 1H), 3.45 (m, 1H), 3.00 (m, 1H), 2.20 (S, 6H) ppm.

### Example 18

(±)-4-(dimethylamino)-N-(2,5-dioxo-7-oxabicyclo[4.1.0]hept-3-en-3-yl)-2-hydroxybenzamide. ¹H NMR. NMR (DMSO-d₆): δ 11.60 (br.s, 1H), 11.00 (br.s, 1H), 7.70 (m, 1H), 6.40 (m, 1H), 6.10 (m, 1H), 4.10 (m, 1H), 3.85 (m, 1H), 3.00 (s, 6H) ppm.

### Example 19

(±)-4-(dimethylamino)-2-hydroxy-N-(2-hydroxy-5-oxo-7-oxabicyclo[4.1.0]hept-3-en-3-yl)benzamide trifluoroacetate. ¹H NMR. NMR (DMSO-d₆): δ11.4 (s, 1H), 10.80 (s, 1H), 7.70 (m, 1H), 6.90 (m, 1H). 6.65 (m, 1H), 6.30 (m, 1H), 6.00 (m, 1H), 4.80 (m, 1H), 3.85 (m, 1H), 3.45 (m, 1H), 2.90 (s, 6H) ppm.

### Example 20

(±)-4-(dimethylamino)-N-(2-hydroxy-5-oxo-7-oxabicyclo[4.1.0]hept-3-en-3-yl)-2-methoxybenzamide. ¹H NMR. NMR (DMSO-d₆): δ11.70 (s, 1H), 7.80 (m, 1H), 6.80 (m, 1H), 6.40 (m, 1H), 6.20 (m, 1H), 4.80 (m, 1H), 3.85 (m, 1H), 3.45 (m, 1H), 3.00 (s, 6H) ppm.

### Example 21

(±)-7-chloro-3-hydroxy-N-(2-hydroxy-5-oxo-7-oxabicyclo[4.1.0]hept-3-en-3-yl)-2-naphthamide. NMR (DMSO-d₆): δ 12.0 (br.s, 1H), 8.60 (m, 1H), 8.15 (m, 1H), 7.70 (m, 1H), 7.50 (m, 1H), 7.25 (m, 1H), 6.95 (m, 1H), 6.70 (m, 1H), 4.90 (m, 1H), 3.85 (m, 1H), 3.45 (m, 1H) ppm.

Employing the general method of Example 1, the following compounds may be prepared:

### Example 22

### (±)-4-tert-butyl-2-hydroxy-N-(2-hydroxy-5-oxo-7-oxabicyclo[4.1.0]hept-3-en-3-yl)benzamide

### Example 23

### (±)-4,5-dichloro-2-hydroxy-N-(2-hydroxy-5-oxo-7-oxabicyclo[4.1.0]hept-3-en-3-yl)benzamide

### Example 24

### (±)-3,6-dichloro-2-hydroxy-N-(2-hydroxy-5-oxo-7-oxabicyclo[4.1.0]hept-3-en-3-yl)benzamide

### Example 25

### (±)-2,3,5-trichloro-6-hydroxy-N-(2-hydroxy-5-oxo-7-oxabicyclo[4.1.0]hept-3-en-3-yl)benzamide

### Example 26

### (±)-5-chloro-2-hydroxy-N-(2-hydroxy-5-oxo-7-oxabicyclo[4.1.0]hept-3-en-3-yl)benzamide

### Example 27

### (±)-2-hydroxy-N-(2-hydroxy-5-oxo-7-oxabicyclo[4.1.0]hept-3-en-3-yl)-5-methylbenzamide

### Results

### Inhibition of NF-κB in Cells by the Compound of Example 10.

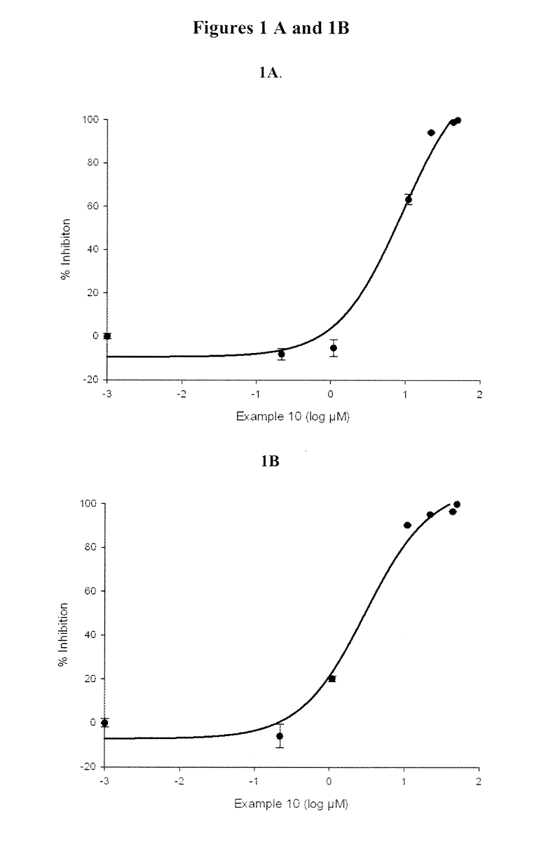

Two reporter cell assays were used to determine the ability of the compound of Example 10 to inhibit NF-κB driven transcription. The first assay was a 293-cell based assay with a stably integrated pNF-κB-luc reporter plasmid containing 3 NF-κB promoter elements. The second assay was a 293-cell based assay with a stably integrated pTRH1-NF-κB-dscGFP reporter containing 4 NF-κB promoter elements. Cells were treated with 0, 0.2, 1, 10, 20 and 40 μM of the compound of Example 10 for 2 hours then were induced with 20 ng/ml TNF-α for 18 hours. Following the induction, luminescence or fluorescence was quantified using a Beckman-Coulter 2300 plate reader. FIGS. 1A and B shows the dose response curve generated from the luminescence and fluorescence data respectively. The compound of Example 10 was observed to inhibit the expression of the luciferase gene in a dose dependent manner with a median IC₅₀ (Inhibitory Concentration of 50%) of 8.7 μM. The compound of Example 10 also inhibited the expression of the Green fluorescent protein gene in a dose dependent manner with a median IC₅₀ of 2.5 μM. These values represent the median value generated from three independent experiments. As a control, 0.5% DMSO treated and untreated cells were compared to verify that the compound of Example 10 had no effect on the expression of luciferase or in the readout of the assay. There was a slight decrease in the output from the assay in the DMSO treated population although it was not statistically significant. As a result of the controls, the decrease in activity in the drug treated samples was compared to the DMSO control sample.

### TransAM NF-κB Family DNA Binding ELISA:

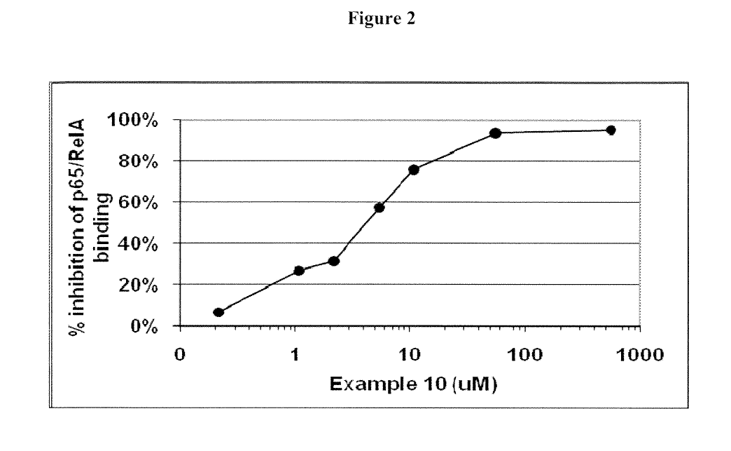

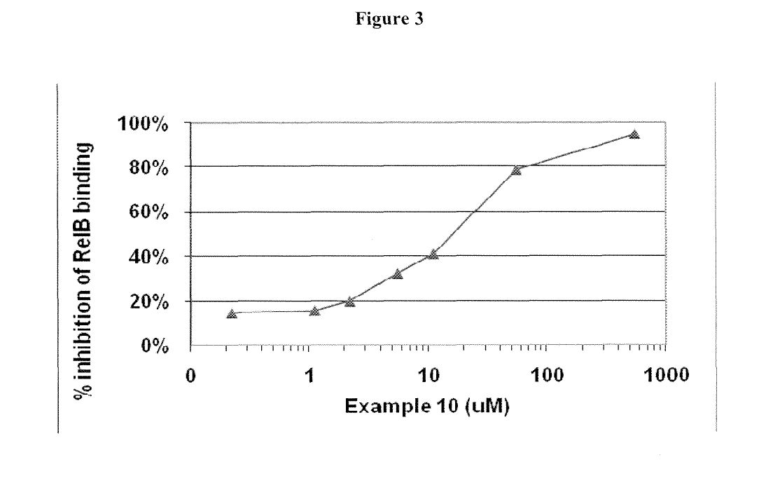

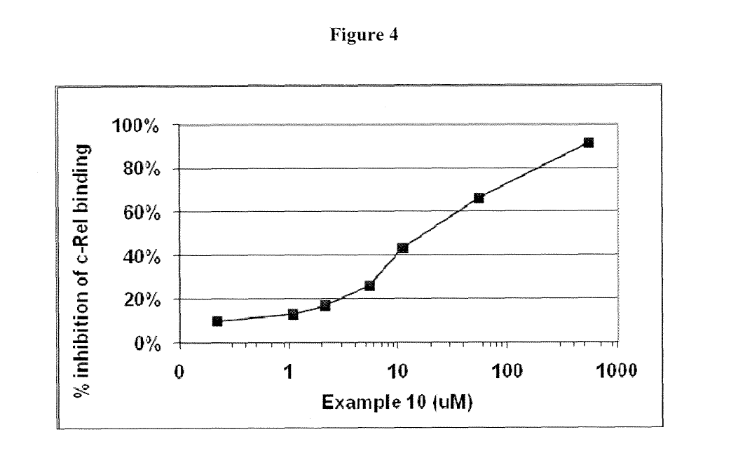

The binding activity of NF-κB heterodimer or homodimer subunits from activated nuclear extracts or purified recombinant NF-κB proteins exposed to the drug compounds was evaluated using the TransAM NF-κB Family binding ELISA (Active Motif). 3-5 μg of nuclear extracts from TNFα activated Hela or Raji cells (Active Motif) or 20 ng of purified recombinant proteins (p65 and p50 from Active Motif, p52 from Santa Cruz) were incubated for 1 hour at room temperature with 20 μL drug compounds diluted in Complete Lysis buffer without DTT. Treated samples were then transferred to 30 μL Complete Binding Buffer (with DTT) in microplate wells pre-coated with the NF-κB consensus oligonucleotide. Controls included non-specific binding (NSB) wells containing lysis buffer without any extract or recombinant protein (for background), nuclear extract or recombinant protein treated with DMSO only (for maximal binding), and wells containing the extract: protein plus 20 pmoles free wild-type NF-κB oligonucleotide as a competitor or 20 pmoles free mutant NF-κB oligonucleotide as a control to demonstrate specificity. The plate was incubated for 1 hour at room temperature with gentle shaking and then washed 3 times with 200 μL 1× Wash Buffer. NF-κB p65, p50, p52, RelB, or c-Rel subunits bound to the plate were detected with 100 μL of the primary antibody (diluted 1:1000 in 1× Antibody Buffer) specific for that subunit. The plate was incubated for 1 hour at room temperature and then washed 3 times with 200 μL 1× Wash Buffer. Next, 100 μL of a HRP conjugated goat anti-rabbit antibody (diluted 1:1,000 in 1× Antibody Buffer) was added to each well. The plate was incubated for 1 hour at room temperature and then washed 4 times with 200 μL 1× Wash Buffer. 100 μL of room temperature Developing Solution was added to each well. The reaction was allowed to develop for 2-10 minutes until a medium dark blue color developed (depending on the subunit activity in the lot of extract or lot of recombinant protein used) and then the reaction was stopped with 100 μL Stop Solution yielding a yellow color. Absorbance was recorded using a Becton-Dickinson DTX 880 Multimode Detector at 450 nm with a reference wavelength subtracted at 620 nm. FIGS. 2, 3 and 4 illustrate the effect of Example 10 on inhibiting RelA, RelB and c-Rel binding to NF-κB sites.

### Inhibition of IL-6 and PGE2 Expressions in RAW264.7.

RAW 264.7 cells were seeded at 4×10⁴ cells per well n complete growth medium in 96 well white TC plates with clear bottoms one day prior to the assay. The next day the cells were washed once and 100 μL fresh growth media was added. Cells were pretreated with 0.5 μL from a 6 point 200× dilution series of the test compounds in DMSO for 2 hours. Following pretreatment with the drugs, the inflammatory response was induced by adding 5 μL of a 20 μg/mL solution of LPS (Sigma). The cells were incubated in the presence of the drugs and 1 μg/mL LPS for another 20-24 hours. Typically after treatment the total DMSO was 0.05% of the culture volume and the final concentrations of the compounds were approximately: 40, 20, 10, 1, 0.2 and 0 μM depending on the MW of each compound. Modified dilution series were prepared as needed to get adequate dose response curves without changing the % DMSO. Samples were run in duplicate or triplicate and included DMSO treated control wells with and without LPS stimulation. Drugs with a known activity such as Parthenolide or DHMEQ were run as experimental controls. After 20-24 hours LPS activation, the media supernatant was collected from the cells and replaced with fresh media. The supernatant samples were cleared by centrifugation at 1,000×g for 5 minutes, transferred to fresh storage plates, and stored frozen at −30° C.

After determining the appropriate supernatant dilutions experimentally, mIL-6 levels in the supernatants were quantified using Quantikine™ mouse IL-6 immunoassay (R&D Systems) according to the manufacturer's protocol. 50 of the supernatants diluted in Calibrator Diluent were added to 50 μL of Assay Diluent in microplate wells pre-coated with an anti-mouse IL-6 capture antibody. Controls included a calibrated positive IL-6 control sample, non-specific binding (NSB) wells containing Calibrator Diluent but no IL-6, and a recombinant mouse IL-6 standard dilution series (10-1000 pg/mL). The plates were incubated at room temperature for 2 hours with shaking and then washed 5 times with 400 μL 1× Wash Buffer. 100 of an HRP-conjugated anti-mouse IL-6 antibody was added to each well to detect IL-6 captured on the plate. The plates were incubated at room temperature for 2 hours and then washed 5 times with 400 μL 1× Wash Buffer. Equal volumes of Color Reagents A and B were mixed and 100 μL of this HRP Substrate Solution was added to each well on the plate. The blue color was allowed to develop for 30 minutes and then the reaction was stopped using 100 μL of Stop Solution yielding a yellow color. Absorbance at 450 nm with a reference wavelength subtracted at 595 nm was recorded using a Becton-Dickinson DTX 880 Multimode Detector.

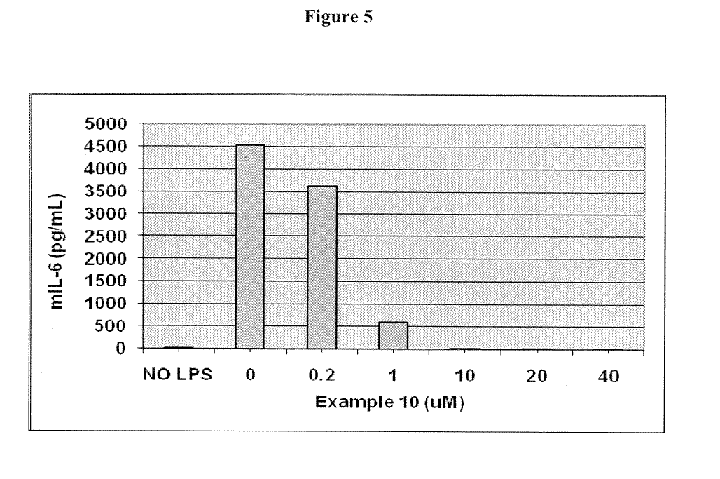

The concentration of mIL-6 in the unknown samples was determined from a curve-fit of the mIL-6 standard absorbance data and multiplying by the dilution factor. The maximum activity achieved in the absence of the inhibitor (DMSO+LPS treated wells) was arbitrarily given a value of 100%; likewise the minimum activity in the absence of the stimulant (no LPS) was assigned a value of 0%. Inhibition of the amount of mIL-6 cytokine released in the drug treated wells was calculated relative to the maximum activation in the DMSO+LPS treated control wells (ie. % inhibition=100−(drug+LPS treated)/(DMSO+LPS treated)). Dose response curves were used to determine the effective concentration to inhibit 50% of the mIL-6 cytokine released (IC₅₀) by means of a SigmaPlot macro which fits a sigmoidal dose-response curve to the (log 10) μM concentration versus % inhibition. In the case when compounds did not reach maximum inhibition at the concentrations tested, the curve fit was assisted with forced maximum (100%) and minimum (0%) values. This technique yields an objective value for the IC₅₀ provided that 50% inhibition was approached at the concentrations tested. FIG. 5 illustrates the effect of the compound of Example 10 on inhibiting IL-6 release.

After determining the appropriate supernatant dilutions experimentally, PGE2 levels in the supernatants were quantified using Parameter™ PGE2 Immunoassay (R&D Systems) according to the manufacturer's protocol. 1004 of the supernatants diluted in Calibrator Diluent and 50 μL of a primary monoclonal anti-PGE2 antibody were added to the microplate wells pre-coated with a goat anti-mouse Ig capture antibody. Then 50 μL of an HRP conjugated PGE2 competitor was added. Controls included non-specific binding (NSB) wells containing Calibrator Diluent but no primary antibody and a recombinant PGE2 standard dilution series (40-5000 pg/mL). The plates were incubated at room temperature for 2 hours with shaking and then washed 5 times with 400 μL 1× Wash Buffer. Equal volumes of Color Reagents A and B were mixed and 200 μL of this HRP Substrate Solution was added to each well on the plate. The blue color was allowed to develop for 30 minutes and then the reaction was stopped using 50 μL of Stop Solution yielding a yellow color. Absorbance at 450 nm with a reference at 595 nm was recorded using a Becton-Dickinson DTX 880 Multimode Detector.

The concentration of PGE2 in the unknown samples was determined from a curve-fit of the PGE2 standard absorbance data and multiplying by the dilution factor. The maximum activity achieved in the absence of the inhibitor (DMSO+LPS treated wells) was arbitrarily given a value of 100%; likewise the minimum activity in the absence of the stimulant (no LPS treated wells) was assigned a value of 0%. Inhibition of the amount of PGE2 released in the drug treated wells was calculated relative to the maximum activation in the DMSO+LPS treated control wells (i.e., % inhibition=100−(drug+LPS treated)/(DMSO+LPS treated)). Dose response curves were used to determine the effective concentration to inhibit 50% of the PGE2 released (IC₅₀) by means of a SigmaPlot macro which fits a sigmoidal dose-response curve to the (log 10) μM concentration versus inhibition. In the case when compounds did not reach maximum inhibition at the concentrations tested, the curve fit was assisted with forced maximum (100%) and minimum (0%) values. This technique yields an objective value for the IC₅₀ provided that 50% inhibition was approached at the concentrations tested.

### Multiple Myeloma Growth Inhibition by Compound 10:

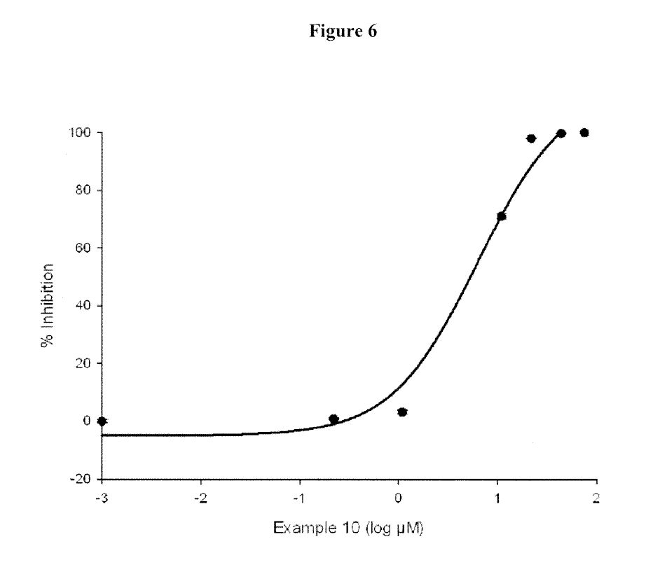

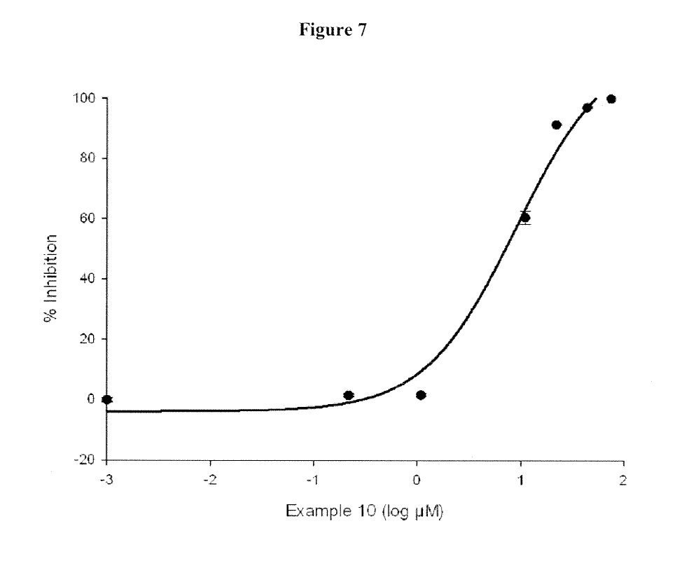

Cryogenically stored RPMI 8226 and U266B1 cells (ATCC, Manassas, Va.) were thawed and cultured according to the vendor's recommendations. Cells were plated in 96-well white-wall tissue culture plates at a concentration of 2×10⁵ cells/ml (0.1 ml per well) and incubated overnight at 37° C. in a humidified 5% CO₂ atmosphere. Compounds were added to each well at the indicated concentrations (0.5 μl of 200×DMSO stock) and incubated with the cells for 48 hours. Cell viability was detected by adding 100 μl CellTiter-Glo Reagent (Promega, Madison, Wis.) to each well, mixing with pipette, and incubating at room temperature for 10 minutes. Luminescence was read with a DTX 880 Multimode Detector (Beckman Coulter, Fullerton, Calif.). Data was analyzed with SigmaPlot 11 (Systat Software, San Jose, Calif.) software. FIGS. 6 and 7 illustrate the effect of the compound of Example 10 on the growth inhibition in multiple myeloma cells.

In addition to the compound of Example 10, Table 1 lists other compounds for their activities to inhibit 1) NF-κB driven expression of luciferase and GFP in HEK293 cells, 2) Il-6 and PGE2 release from RAW264 cells, and 3) binding of RelA, RelB, c-Rel to NF-κB sites.

TABLE 1
Pharmacological activities of compounds in inhibition of NF-κB driven
reporter gene expression, suppression of cytokine release and inhibition
of Rel protein bindings to NF-κB sites.

| | | NF- | RAW | RAW | | c-Rel | RelB |
|---|---|---|---|---|---|---|---|
| | 293/ | KB/ | 264.7 | 264.7 | | binding | binding |
| | NFkB- | 293/ | IL-6 | PGE2 | p65 | IC50 (uM) | IC50 (uM) |
| | luc | GFP | release | release | binding | or % | or % |
| Ex. | EC50 | EC50 | EC50 | EC50 | IC50 | inhibition | inhibition |
| # | (uM) | (uM) | (uM) | (uM) | (uM) | at 5 uM | at 5 uM |
| 1 | 36 | 3.4 | 3.1 | 13.3 | 66.3 | 1% | 6% |
| 2 | 12 | 7.0 | 1.3 | 5.9 | 26.3 | 7% | 18% |
| 3 | 20 | 9 | 0.48 | 10 | 17 | 5% | 10% |
| 4 | 19 | 18 | 0.76 | 2.6 | 36.3 | 0% | 9% |
| 5 | 32 | 6.1 | 0.42 | 5.7 | 14.2 | 6% | 17% |
| 6 | 37 | 14 | 2.5 | 7.2 | 19.4 | 7% | 13% |
| 7 | 21 | 4.7 | 1.4 | 4.6 | 488 | 2% | 6% |
| 8 | 7.1 | 7.1 | 0.30 | N/D | 45.2 | 3% | 15% |
| 9 | 27 | 5.2 | 6.5 | N/D | 17.7 | 20% | 32% |
| 10 | 8.7 | 2.5 | 0.44 | N/D | 3.9 | 17 | 23 |
| 11 | 7.7 | 5.0 | 0.85 | N/D | 479 | 16 | 10 |
| 12 | 2.2 | 3.6 | 0.14 | N/D | 9.7 | N/D | N/D |
| 13 | 21 | 9.2 | 1.7 | N/D | 8.7 | N/D | N/D |
| 14 | 2.9 | 3.6 | 0.25 | N/D | 5.3 | N/D | N/D |
| 15 | 8.0 | 2.4 | 0.22 | N/D | 241 | 3.7 | 3.9 |
| 16 | 8.7 | 2.6 | 0.27 | N/D | 1.5 | 8.1 | 2.1 |
| 17 | 3.7 | 6.7 | 0.48 | N/D | 295 | 232 | 82 |
| 18 | 2.1 | 3.1 | 0.58 | N/D | 26 | N/D | N/D |
| 19 | 7.5 | 1.7 | 0.74 | N/D | 3.0 | 14 | 3.3 |
| 20 | 4.6 | 1.3 | 0.24 | N/D | 0.91 | 1.9 | 1.0 |

Without further description, it is believed that one of ordinary skill in the art can, using the preceding description and the following illustrative examples, make and utilize the compounds of the present invention and practice the claimed methods. While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. All patents, patent applications and other references cited throughout this application are herein incorporated by reference in their entirety.

### REFERENCES

Aggarwal B B, Shishodia S, Sandur S K, Pandey M K, Sethi G (2006) Inflammation and cancer: how hot is the link? Biochem Pharmacol 72(11): 1605-1621.
Arcaroli J, Silva E, Maloney J P, He Q, Svetkauskaite D, Murphy J R, Abraham E (2006) Variant IRAK-1 haplotype is associated with increased nuclear factor-kappaB activation and worse outcomes in sepsis. Am J Respir Crit. Care Med 173(12): 1335-1341.
Argyropoulos C, Mouzaki A (2006) Immunosuppressive drugs in HIV disease. Curr Top Med Chem 6(16): 1769-1789.
Ariga A, Namekawa J-i, Matsumoto N, Inoue J-i, Umezawa K (2002) Inhibition of tumor necrosis factor Oalpha-induced nuclear translocation and activation of NF-kappaB by dehydroxymethylepoxyquinomicin. J. Biol. Chem. 277(27): 24625-24630.
Atreya I, Atreya R, Neurath M F (2008) NF-kappaB in inflammatory bowel disease. J Intern Med 263(6): 591-596.
Baba M (2006) Recent status of HIV-1 gene expression inhibitors. Antiviral Res 71(2-3): 301-3Bames P J (1997) Nuclear factor-kappa B. Int J Biochem Cell Biol 29(6): 867-870.
Bosisio D, Marazzi I, Agresti A, Shimizu N, Bianchi M E, Natoli G (2006) A hyper-dynamic equilibrium between promoter-bound and nucleoplasmic dimers controls NF-kappaB-dependent gene activity. Embo J 25(4): 798-810.
Bouma G, Strober W (2003) The immunological and genetic basis of inflammatory bowel disease. Nat Rev Immunol 3(7): 521-533.
Burstein E, Duckett C S (2003) Dying for NF-kappaB? Control of cell death by transcriptional regulation of the apoptotic machinery. Curr Opin Cell Biol 15(6): 732-737.
Calzado M A, Bacher S, Schmitz M L (2007) NF-kappaB inhibitors for the treatment of inflammatory diseases and cancer. Curr Med Chem 14(3): 367-376.
Chaicharoenpong C, Kato K, Umezawa K (2002) Synthesis and structure-activity relationship of dehydroxymethylepoxyquinomicin analogues as inhibitors of NF-kappaB functions. Bioorg Med Chem 10(12): 3933-3999.
Flory E, Kunz M, Scheller C, Jassoy C, Stauber R, Rapp U R, Ludwig S (2000) Influenza virus-induced NF-kappaB-dependent gene expression is mediated by overexpression of viral proteins and involves oxidative radicals and activation of IkappaB kinase. J Biol Chem 275(12): 8307-8314.
Giri R K, Rajagopal V, Shahi S, Zlokovic B V, Kalra V K (2005) Mechanism of amyloid peptide induced CCR5 expression in monocytes and its inhibition by siRNA for Egr-1. Am J Physiol Cell Physiol 289(2): C264-276.
Goldring S R, Jasty N I, Roelke M S, Rourke C M, Bringhurst F R, Harris W H (1986) Formation of a synovial-like membrane at the bone-cement interface. Its role in bone resorption and implant loosening after total hip replacement. Arthritis Rheum 29(7): 836-842.
Greten F R, Karin M (2004) The IKKNF-kappaB activation pathway-a target for prevention and treatment of cancer. Cancer Lett 206(2): 193-199.
Hacker H, Karin M (2006) Regulation and function of IKK and IKK-related kinases. Sci STKE 2006(357): re13.
Harris W H (1995) The problem is osteolysis. Clin Orthop Relat Res (311): 46-53.
Hayden M S, Ghosh S (2004) Signaling to NF-kappaB. Genes Dev 18(18): 2195-2224.
Hayden M S, West A P, Ghosh S (2006a) NF-kappaB and the immune response. Oncogene 25(51): δ758-6780.
Hayden M S, West A P, Ghosh S (2006b) SnapShot: NF-kappaB Signaling Pathways. Cell 127(6): 1286-1287.
Helbig G, Christopherson K W, 2nd, Bhat-Nakshatri P, Kumar S, Kishimoto H, Miller K D, Broxmeyer H E, Nakshatri H (2003) NF-kappaB promotes breast cancer cell migration and metastasis by inducing the expression of the chemokine receptor CXCR4. J Biol Chem 278(24): 21631-21638.
Iordanskiy S, Iordanskaya T, Quivy V, Van Lint C, Bukrinsky M (2002) B-oligomer of pertussis toxin inhibits HIV-1 LTR-driven transcription through suppression of NF-kappaB p65 subunit activity. Virology 302(1): 195-206.
Israel A (2000) The IKK complex: an integrator of all signals that activate NF-kappaB? Trends Cell Biol 10(4): 129-133.
Jung M, Zhang Y, Lee S, Dritschilo A (1995) Correction of radiation sensitivity in ataxia telangiectasia cells by a truncated I kappa B-alpha. Science 268(5217): 1619-1621.
Kalergis A M, Iruretagoyena M I, Barrientos M J, Gonzalez P A, Herrada A A, Leiva E D, Gutierrez M A, Riedel C A, Bueno S M, Jacobelli S H (2008) Modulation of nuclear factor-kappaB activity can influence the susceptibility to systemic upus erythematosus. Immunology.
Karin M (1999) How NF-kappaB is activated: the role of the IkappaB kinase (IKK) complex. Oncogene 18(49): δ867-6874.
Karin M (2006) Nuclear factor-kappaB in cancer development and progression. Nature 441(7092): 431-436.
Ketas T J, Kuhmann S E, Palmer A, Zurita J, He W, Ahuja S K, Klasse P J, Moore J P (2007) Cell surface expression of CCR5 and other host factors influence the inhibition of HIV-1 infection of human lymphocytes by CCR5 ligands. Virology 364(2):281-90.
Kurylowicz A, Nauman J (2008) The role of nuclear factor-kappaB in the development of autoimmune diseases: a link between genes and environment. Acta Biochim Pol 55(4): 629-647.
Lawrence R C, Helmick C G, Arnett F C, Deyo R A, Felson D T, Giannini E H, Heyse S P, Hirsch R, Hochberg M C, Hunder G G, Liang M H, Pillemer S R, Steen V D, Wolfe F (1998) Estimates of the prevalence of arthritis and selected musculoskeletal disorders in the United States. Arthritis Rheum 41(5): 778-799.
Liao F, Andalibi A, Qiao J H, Allayee H, Fogelman A M, Lusis A J (1994) Genetic evidence for a common pathway mediating oxidative stress, inflammatory gene induction, and aortic fatty streak formation in mice. J Clin Invest 94(2): 877-884.
Liu R, Zhao X, Gurney T A, Landau N R (1998) Functional analysis of the proximal CCR5 promoter. AIDS Res Hum Retroviruses 14(17): 1509-1519.
Maruyama I, Shigeta K, Miyahara H, Nakajima T, Shin H, Ide S, Kitajima I (1997) Thrombin activates NF-kappa B through thrombin receptor and results in proliferation of vascular smooth muscle cells: role of thrombin in atherosclerosis and restenosis. Ann N Y Acad Sci 811: 429-436.
Mukerjee R, Sawaya B E, Khalili K, Amini S (2006) Association of p65 and C/EBPbeta with HIV-1 LTR modulates transcription of the viral promoter. J Cell Biochem 100(5):1210-6.
Natoli G, Saccani S, Bosisio D, Marazzi I (2005) Interactions of NF-kappaB with chromatin: the art of being at the right place at the right time. Nat Immunol 6(5): 439-445.
Neurath M F, Fuss I, Schurmann G, Pettersson S, Arnold K, Muller-Lobeck H, Strober W, Herfarth C, Buschenfelde K H (1998) Cytokine gene transcription by NF-kappa B family members in patients with inflammatory bowel disease. Ann N Y Acad Sci 859: 149-159.
Niu J, Azfer A, Kolattukudy P E (2008) Protection against lipopolysaccharide-induced myocardial dysfunction in mice by cardiac-specific expression of soluble Fas. J Mol Cell Cardiol 44(1): 160-169.
Pahl H L, Baeuerle P A (1995) Expression of influenza virus hemagglutinin activates transcription factor NF-kappa B. J Virol 69(3): 1480-1484.
Palmieri C, Trimboli F, Puca A, Fiume G, Scala G, Quinto I (2004) Inhibition of HIV-1 replication in primary human monocytes by the IkappaB-alphaS32/36A repressor of NF-kappaB. Retrovirology 1(1): 45.
Pihlstrom B L, Michalowicz B S, Johnson N W (2005) Periodontal diseases. Lancet 366(9499): 1809-1820.
Platt E J, Wehrly K, Kuhmann S E, Chesebro B, Kabat D (1998) Effects of CCR5 and CD4 cell surface concentrations on infections by macrophagetropic isolates of human immunodeficiency virus type 1. J Virol 72(4): 2855-2864.
Reeves J D, Gallo S A, Ahmad N, Miamidian J L, Harvey P E, Sharron M, Pohlmann S, Sfakianos T N, Derdeyn C A, Blumenthal R, Hunter E, Doms R W (2002) Sensitivity of HIV-1 to entry inhibitors correlates with envelope/coreceptor affinity, receptor density, and fusion kinetics. Proc Natl Acad Sci USA 99(25): 16249-16254.
Rizzi C, Alfano M, Bugatti A, Camozzi M, Poli G, Rusnati M (2004) Inhibition of intra- and extra-cellular Tat function and HIV expression by pertussis toxin B-oligomer. Eur J Immunol 34(2): 530-536.
Saccani S, Marazzi I, Beg A A, Natoli G (2004) Degradation of promoter-bound p65/RelA is essential for the prompt termination of the nuclear factor kappaB response. J Exp Med 200(1): 107-113.
Scheidereit C (2006) IkappaB kinase complexes: gateways to NF-kappaB activation and transcription. Oncogene 25(51): 6685-6705.
Sui Z, Sniderhan L F, Fan S, Kazmierczak K, Reisinger E, Kovacs A D, Potash M J, Dewhurst S, Gelbard H A, Maggirwar S B (2006) Human immunodeficiency virus-encoded Tat activates glycogen synthase kinase-3beta to antagonize nuclear factor-kappaB survival pathway in neurons. Eur J Neurosci 23(10): 2623-2634.
Takeuich T, Umezawa K, To-E S, Matsumoto N, Sawa T, Yoshioka T, Agata N, Hirano S-i, Isshiki K (2003) Salicylamide deratives. U.S. Pat. No. 6,566,394 B1.
Tergaonkar V (2006) NFkappaB pathway: a good signaling paradigm and therapeutic target. Int J Biochem Cell Biol 38(10): 1647-1653.
Tilg H, Moschen A R, Kaser A, Pines A, Dotan I (2008) Gut, inflammation and osteoporosis: basic and clinical concepts. Gut 57(5): δ84-694.
Suzuki Y, Sugiyama C, Ohno O, Umezawa K (2004) Preparation and biological activities of optically active dehydroxymethylepoxyquinomicin, a novel NF-kB inhibitor. Tetrahedron 60:7061-7066.
Umezawa K (2006) Inhibition of tumor growth by NF-kappaB inhibitors. Cancer Sci 97(10):990-995.
Williams S A, Chen L F, Kwon H, Ruiz-Jarabo C M, Verdin E, Greene W C (2006) NF-kappaB p50 promotes HIV latency through HDAC recruitment and repression of transcriptional initiation. EMBO J25(1): 139-149.
Williams S A, Kwon H, Chen L F, Greene W C (2007) Sustained Induction of NF-{kappa} B Is Required for Efficient Expression of Latent HIV-1. J Virol 81(11):6043-56.
Wirtz S, Neurath M F (2007) Mouse models of inflammatory bowel disease. Adv Drug Deily Rev 59(11): 1073-1083.
Yamamoto M, Horie R, Takeiri M, Kozawa I, Umezawa K (2008) Inactivation of NF-kappaB components by covalent binding of (−)-dehydroxymethylepoxyquinomicin to specific cysteine residues. J. Med Chem 51(8):5780-5788.

1. A compound of formula (1) or formula (2): 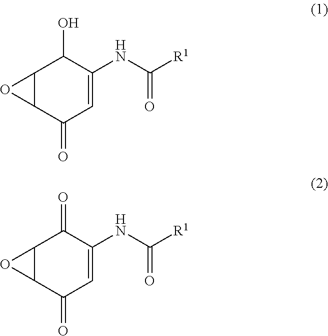
or a pharmaceutically acceptable salt thereof, wherein
R¹ is 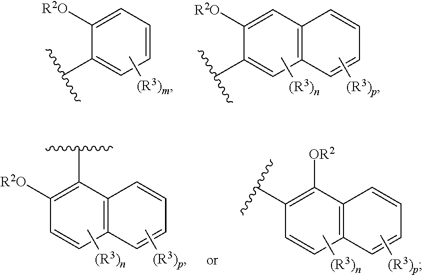
each R³ is independently hydrogen; CF₃; phenyl optionally substituted with cyano, halo, nitro, hydroxyl, (C1-C6)alkyl, (C1-C6)alkyl-OH, (C1-C6)alkoxy, COR⁴, NR⁵R⁶ or NHCO(C1-C6)alkyl; cyano; halo; nitro; hydroxyl; (C1-C6)alkyl; (C3-C6)cycloalkyl; (C1-C6)alkyl-OH; (C1-C6)-alkyl-NR⁵R⁶; trifluoromethyl; (C1-C6)alkoxy; (C1-C6)thioalkoxy; phenoxy; COR⁴; NR⁵R⁶; NHCO(C1-C6)alkyl; SO₃H; SO₂(C1-C6)alkyl or SO₂NR⁵R⁶, wherein at least one R³ is other than hydrogen when R¹ is phenyl;
R² is H; R⁷; COR⁷; CONHR⁷; COOR⁷; CH₂OCOR⁷; P(O)(OH)₂; P(O)(O(C1-C6)alkyl)₂; P(O)(OCH₂OCO(C1-C6)alkyl)₂; P(O)(OH)(OCH₂OCO(C1-C6)alkyl); P(O)(OH)(O(C1-C6)alkyl), P(O)(OH)(C1-C6)alkyl, an inorganic salt of P(O)(O(C1-C6)alkyl)₂, P(O)(OCH₂OCO(C1-C6)alkyl)₂, P(O)(OH)(OCH₂OCO(C1-C6)alkyl), P(O)(OH)(O(C1-C6)alkyl), or P(O)(OH)(C1-C6)alkyl); or glycosyl;
R⁷ is (C1-C6)alkyl, trifluoromethyl, (C3-C6)cycloalkyl, cyclohexylmethyl or phenyl, wherein the phenyl is substituted with 0 to 4 groups selected from the group consisting of fluorine, chlorine, bromine, hydroxyl, trifluoromethyl, (C1-C4)alkyl, (C1-C4)alkoxy, phenylmethyl, and phenylmethyl optionally substituted with fluorine, chlorine, bromine, hydroxyl, trifluoromethyl, (C1-C4)alkyl, (C1-C4)alkoxy, 2-pyridinyl, 3-pyridinyl, or 4-pyridinyl, 2-pyrimidinyl, 4-pyrimidinyl, or 5-pyrimidinyl;
R⁴ is independently hydroxyl, (C1-C6)alkoxy, phenoxy or —NR⁵R⁶;
R⁵ and R⁶ are independently hydrogen; (C1-C6)alkyl or (C3-C6)cycloalkyl; or
R⁵ and R⁶ combine, along with the nitrogen atom, to form a six-membered ring containing an additional heteroatom selected from the group consisting of nitrogen, oxygen and sulfur, wherein the ring is optionally substituted with (C1-C6)alkyl or (C3-C6)cycloalkyl);
m is 1 to 4; and
n+p is 0 to 6.

2. The compound of claim 1, wherein R² is H.

3. The compound of claim 1, wherein R¹ is

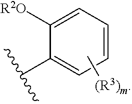

4. The compound of claim 1, wherein R¹ is

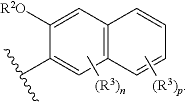

5. The compound of claim 1, wherein R¹ is

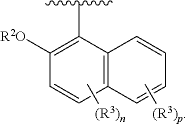

6. The compound of claim 1, wherein R¹ is

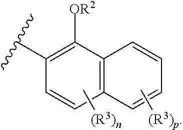

7. The compound of claim 1, wherein R² is H.

8. The compound of claim 1, wherein:
R² is R⁷; COR⁷; CONHR⁷; COOR⁷; CH₂OCOR⁷; P(O)(OH)₂; P(O)(O(C1-C6)alkyl)₂; P(O)(OCH₂OCO(C1-C6)alkyl)₂; P(O)(OH)(OCH₂OCO(C1-C6)alkyl); P(O)(OH)(O(C1-C6)alkyl); P(O)(OH)(C1-C6)alkyl); an inorganic salt of P(O)(O(C1-C6)alkyl)₂, P(O)(OCH₂OCO(C1-C6)alkyl)₂, P(O)(OH)(OCH₂OCO(C1-C6)alkyl), P(O)(OH)(O(C1-C6)alkyl), or P(O)(OH)(C1-C6)alkyl; or glycosyl;
R⁷ is (C1-C6)alkyl, trifluoromethyl, cyclopropyl, cyclohexyl, cyclohexylmethyl or phenyl, wherein the phenyl is substituted with 0 to 4 groups selected from the group consisting of fluorine, chlorine, bromine, hydroxyl, trifluoromethyl, (C1-C4)alkyl, (C1-C4)alkoxy, phenylmethyl, phenylmethyl substituted with fluorine, chlorine, bromine, hydroxyl, trifluoromethyl, (C1-C4)alkyl, (C1-C4)alkoxy, 2-pyridinyl, 3-pyridinyl, 4-pyridinyl, 2-pyrimidinyl, 4-pyrimidinyl, and 5-pyrimidinyl.

9. The compound of claim 1, having the structure of formula (3) 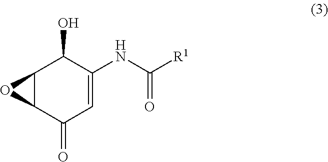
.

10. The compound of claim 9, wherein R¹ is

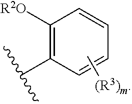

11. The compound of claim 9, wherein R¹ is

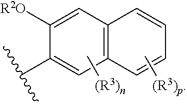

12. The compound of claim 9, wherein R¹ is

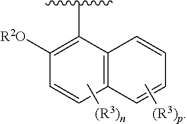

13. The compound of claim 9, wherein R¹ is

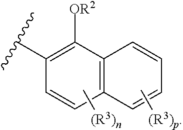

14. The compound claim 10, wherein R² is H.

15. The compound of claim 10, wherein:
R² is R⁷, COR⁷, CONHR⁷, COOR⁷, CH₂OCOR⁷, P(O)(OH)₂, P(O)(O(C1-C6)alkyl)₂, P(O)(OCH₂OCO(C1-C6)alkyl)₂, P(O)(OH)(OCH₂OCO(C1-C6)alkyl), P(O)(OH)(O(C1-C6)alkyl), P(O)(OH)(C1-C6)alkyl, an inorganic salt of P(O)(O(C1-C6)alkyl)₂, P(O)(OCH₂OCO(C1-C6)alkyl)₂, P(O)(OH)(OCH₂OCO(C1-C6)alkyl), P(O)(OH)(O(C1-C6)alkyl), or P(O)(OH)(C1-C6)alkyl, or glycosyl,
R⁷ is (C1-C6)alkyl, trifluoromethyl, cyclopropyl, cyclohexyl, cyclohexylmethyl or phenyl, wherein the phenyl is substituted with 0 to 4 groups selected from the group consisting of fluorine, chlorine, bromine, hydroxyl, trifluoromethyl, (C1-C4)alkyl, (C1-C4)alkoxy, phenylmethyl, and phenylmethyl substituted with fluorine, chlorine, bromine, hydroxyl, trifluoromethyl, (C1-C4)alkyl, (C1-C4)alkoxy, 2-pyridinyl, 3-pyridinyl, 4-pyridinyl, 2-pyrimidinyl, 4-pyrimidinyl, or 5-pyrimidinyl.

16. A pharmaceutical composition comprising a compound according to claim 1 or a pharmaceutically acceptable salt thereof in combination with a pharmaceutically effective diluent or carrier.

17. A method of treating a disease in a mammal associated with inhibition of activation of NF-κB, comprising administering to a mammal in need thereof a compound of formula (1) or formula (2), or a pharmacologically acceptable salt thereof according to claim 1.

18. The method of claim 17, wherein the disease is selected from the group consisting of cancer, inflammation, auto-immune diseases, diabetes and diabetic complications, infection, cardiovascular disease and ischemia-reperfusion injuries.

19. The compound of claim 1, which is selected from the group consisting of:
2-hydroxy-N-(2-hydroxy-5-oxo-7-oxabicyclo[4.1.0]hept-3-en-3-yl)-4-(trifluoromethyl)benzamide;
(±)-4-chloro-2-hydroxy-N-(2-hydroxy-5-oxo-7-oxabicyclo[4.1.0]hept-3-en-3-yl)benzamide;
(±)-2-hydroxy-N-(2-hydroxy-5-oxo-7-oxabicyclo[4.1.0]hept-3-en-3-yl)-4-methylbenzamide;
(±)-2-hydroxy-N-(2-hydroxy-5-oxo-7-oxabicyclo[4.1.0]hept-3-en-3-yl)-3-methylbenzamide;
(±)-2-hydroxy-N-(2-hydroxy-5-oxo-7-oxabicyclo[4.1.0]hept-3-en-3-yl)-4-methoxybenzamide;
(±)-3-chloro-2-hydroxy-N-(2-hydroxy-5-oxo-7-oxabicyclo[4.1.0]hept-3-en-3-yl)benzamide;
(±)-3-hydroxy-N-(2-hydroxy-5-oxo-7-oxabicyclo[4.1.0]hept-3-en-3-yl)-2-naphthamide;
(±)-1-hydroxy-N-(2-hydroxy-5-oxo-7-oxabicyclo[4.1.0]hept-3-en-3-yl)-2-naphthamide;
(±)-5-bromo-2-hydroxy-N-(2-hydroxy-5-oxo-7-oxabicyclo[4.1.0]hept-3-en-3-yl)benzamide;
(±)-4-(dimethylamino)-2-hydroxy-N-(2-hydroxy-5-oxo-7-oxabicyclo[4.1.0]hept-3-en-3-yl)benzamide;
(±)-3-hydroxy-N-(2-hydroxy-5-oxo-7-oxabicyclo[4.1.0]hept-3-en-3-yl)-7-methoxy-2-naphthamide;
(±)-N-(2,5-dioxo-7-oxabicyclo[4.1.0]hept-3-en-3-yl)-2-hydroxy-1-naphthamide;
(±)-5-bromo-2-hydroxy-N-(2-hydroxy-5-oxo-7-oxabicyclo[4.1.0]hept-3-en-3-yl)-3-methylbenzamide;
(±)-5-bromo-N-(2,5-dioxo-7-oxabicyclo[4.1.0]hept-3-en-3-yl)-2-hydroxy-3-methylbenzamide;
(±)-7-bromo-3-hydroxy-N-(2-hydroxy-5-oxo-7-oxabicyclo[4.1.0]hept-3-en-3-yl)-2-naphthamide;
(±)-3,4-difluoro-N-(2-hydroxy-5-oxo-7-oxabicyclo[4.1.0]hept-3-en-3-yl)-2-methoxybenzamide;
(±)-2-hydroxy-N-(2-hydroxy-5-oxo-7-oxabicyclo[4.1.0]hept-3-en-3-yl)-6-isopropyl-3-methylbenzamide;
(±)-4-(dimethylamino)-N-(2,5-dioxo-7-oxabicyclo[4.1.0]hept-3-en-3-yl)-2-hydroxybenzamide;
(±)-4-(dimethylamino)-2-hydroxy-N-(2-hydroxy-5-oxo-7-oxabicyclo[4.1.0]hept-3-en-3-yl)benzamide trifluoroacetate;
(±)-4-(dimethylamino)-N-(2-hydroxy-5-oxo-7-oxabicyclo[4.1.0]hept-3-en-3-yl)-2-methoxybenzamide;
(±)-7-chloro-3-hydroxy-N-(2-hydroxy-5-oxo-7-oxabicyclo[4.1.0]hept-3-en-3-yl)-2-naphthamide;
(±)-4-tert-butyl-2-hydroxy-N-(2-hydroxy-5-oxo-7-oxabicyclo[4.1.0]hept-3-en-3-yl)benzamide;
(±)-4,5-dichloro-2-hydroxy-N-(2-hydroxy-5-oxo-7-oxabicyclo[4.1.0]hept-3-en-3-yl)benzamide;
(±)-3,6-dichloro-2-hydroxy-N-(2-hydroxy-5-oxo-7-oxabicyclo[4.1.0]hept-3-en-3-yl)benzamide;
(±)-2,3,5-trichloro-6-hydroxy-N-(2-hydroxy-5-oxo-7-oxabicyclo[4.1.0]hept-3-en-3-yl)benzamide;
(±)-5-chloro-2-hydroxy-N-(2-hydroxy-5-oxo-7-oxabicyclo[4.1.0]hept-3-en-3-yl)benzamide; and
(±)-2-hydroxy-N-(2-hydroxy-5-oxo-7-oxabicyclo[4.1.0]hept-3-en-3-yl)-5-methylbenzamide.

20. A method of treating a disease in a mammal associated with inhibition of activation of NF-κB, comprising administering to a mammal in need thereof a compound of claim 19.
